(19) United States
(12) Patent Application Publication
(10) Pub. No.: US 2003/0078517 A1
(43) Pub. Date: Apr. 24, 2003

(54) In vivo delivery methods and compositions

(76) Inventor: Kenneth Kensey, Malvern, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 09/839,785

(22) Filed: Apr. 20, 2001

Related U.S. Application Data
(63) Continuation-in-part of application No. 09/819,924, filed on Mar. 28, 2001.
Application No. 09/819,924 is a continuation-in-part of application No. 09/727,950, filed on Dec. 1, 2000, now abandoned.
Application No. 09/727,950 is a continuation-in-part of application No. 09/628,401, filed on Aug. 1, 2000.
Application No. 09/628,401 is a continuation-in-part of application No. 09/501,856, filed on Feb. 10, 2000, now Pat. No. 6,322,525.
Application No. 09/501,856 is a continuation-in-part of application No. 09/439,795, filed on Nov. 12, 1999, now Pat. No. 6,322,524.
Application No. 09/439,795 is a continuation-in-part of application No. 08/919,906, filed on Aug. 28, 1997, now Pat. No. 6,019,735.

(51) Int. Cl. A61M 31/00; A61B 5/02; A61B 5/00; B65D 81/00

(52) U.S. Cl. 600/573; 604/66; 604/67; 600/504

(57) ABSTRACT

Various methods are provided for determining and utilizing the viscosity of the circulating blood of a living being over a range of shear rates for diagnostics and treatment, such as detecting/reducing blood viscosity, work of the heart, contractility of the heart, for detecting/reducing the surface tension of the blood, for detecting plasma viscosity, for explaining/countering endothelial cell dysfunction, for providing high and low blood vessel wall shear stress data, red blood cell deformability data, lubricity of blood, and for treating different ailments such as peripheral arterial disease in combination with administering to a living being at least one pharmaceutically acceptable agent. Agents pharmaceutically effective to regulate at least one of the aforementioned blood parameters are used to adjust distribution of a substance through the bloodstream.

### RELATED APPLICATIONS

This application is Continuation-in-Part of the copending U.S. application filed Apr. 9, 2001 entitled IN VIVO DELIVERY METHODS AND COMPOSITIONS and having the identifying Attorney Docket No. V1025/20099, which application in turn is a Continuation-in-Part of the copending U.S. application Ser. No. 09/819,924 filed Mar. 28, 2001 entitled IN VIVO DELIVERY METHODS AND COMPOSITIONS, which application in turn is a Continuation-in-Part of co-pending U.S. patent application Ser. No. 09/727,950, filed Dec. 1, 2000, entitled METHODS AND COMPOSITIONS FOR ADJUSTING PHYSICAL PROPERTIES OF CIRCULATING BLOOD, which in turn is a Continuation-in-Part of co-pending U.S. patent application Ser. No. 09/628,401, filed Aug. 1, 2000, entitled APPARATUS & METHODS FOR COMPREHENSIVE BLOOD ANALYSIS, INCLUDING WORK OF, AND CONTRACTILITY OF, HEART, AND THERAPEUTIC APPLICATIONS AND COMPOSITIONS THEREOF, which in turn is a Continuation-in-Part of Co-Pending application Ser. No. 09/501,856, filed Feb. 10, 2000, which in turn is a Continuation-in-Part of Co-Pending application Ser. No. 09/439,795, filed Nov. 12, 1999, entitled DUAL RISER/SINGLE CAPILLARY VISCOMETER, which in turn is a Continuation-in-Part of Co-Pending application Ser. No. 08/919,906, filed Aug. 28, 1997 (now U.S. Pat. No. 6,019,735, issued on Feb. 1, 2000), entitled VISCOSITY MEASURING APPARATUS AND METHOD OF USE, all of which are assigned to the same Assignee as the present invention and all of whose entire disclosures are incorporated by reference herein.

### BACKGROUND OF THE INVENTION

This invention relates generally to apparatus and methods for determining and utilizing the viscosity of the circulating blood of a living being for diagnostics and treatment, and more particularly, apparatus and methods for detecting/reducing blood viscosity, work of the heart, contractility of the heart, for detecting/reducing the surface tension of the blood, for detecting plasma viscosity, for explaining/countering endothelial cell dysfunction, for providing high and low blood vessel wall shear stress data, red blood cell deformability, lubricity of blood, and for treating different ailments, such as peripheral arterial disease.

The importance of determining the viscosity of blood is well-known. Fibrogen, Viscosity and White Blood Cell Count Are Major Risk Factors for Ischemic Heart Disease, by Yarnell et al., Circulation, Vol. 83, No. 3, March 1991; Postprandial Changes in Plasma and Serum Viscosity and Plasma Lipids and Lipoproteins After an Acute Test Meal, by Tangney, et al., American Journal for Clinical Nutrition, 65:36-40, 1997; Studies of Plasma Viscosity in Primary Hyperlipoproteinaemia, by Leonhardt et al., Atherosclerosis 28, 29-40, 1977; Effects of Lipoproteins on Plasma Viscosity, by Seplowitz, et al., Atherosclerosis 38, 89-95, 1981; Hyperviscosity Syndrome in a Hypercholesterolemic Patient with Primary Biliary Cirrhosis, Rosenson, et al., Gastroenterology, Vol. 98, No. 5, 1990; Blood Viscosity and Risk of Cardiovascular Events: the Edinburgh Artery Study, by Lowe et al., British Journal of Hematology, 96, 168-171, 1997; Blood Rheology Associated with Cardiovascular Risk Factors and Chronic Cardiovascular Diseases: Results of an Epidemiologic Cross-Sectional Study, by Koenig, et al., Angiology, The Journal of Vascular Diseases, November 1988; Importance of Blood Viscoelasticity in Arteriosclerosis, by Hell, et al., Angiology, The Journal of Vascular Diseases, June, 1989; Thermal Method for Continuous Blood-Velocity Measurements in Large Blood Vessels, and Cardiac-Output Determination, by Delanois, Medical and Biological Engineering, Vol. 11, No. 2, March 1973; Fluid Mechanics in Atherosclerosis, by Nerem, et al., Handbook of Bioengineering, Chapter 21, 1985.

Much effort has been made to develop apparatus and methods for determining the viscosity of blood. Theory and Design of Disposable Clinical Blood Viscometer, by Litt et al., Biorheology, 25, 697-712, 1988; Automated Measurement of Plasma Viscosity by Capillary Viscometer, by Cooke, et al., Journal of Clinical Pathology 41, 1213-1216, 1988; A Novel Computerized Viscometer/Rheometer by Jimenez and Kostic, Rev. Scientific Instruments 65, Vol 1, January 1994; A New Instrument for the Measurement of Plasma-Viscosity, by John Harkness, The Lancet, pp. 280-281, Aug. 10, 1963; Blood Viscosity and Raynaud's Disease, by Pringle, et al., The Lancet, pp. 1086-1089, May 22, 1965; Measurement of Blood Viscosity Using a Conicylindrical Viscometer, by Walker et al., Medical and Biological Engineering, pp. 551-557, September 1976.

One reference, namely, The Goldman Algorithm Revisited: Prospective Evaluation of a Computer-Derived Algorithm Versus Unaided Physician Judgment in Suspected Acute Myocardial Infarction, by Qamar, et al., Am HeartJ 138(4):705-709, 1999, discusses the use of the Goldman algorithm for providing an indicator to acute myocardial infarction. The Goldman algorithm basically utilizes facts from a patient's history, physical examination and admission (emergency room) electrocardiogram to provide an AMI indicator.

In addition, there are a number of patents relating to blood viscosity measuring apparatus and methods. See for example, U.S. Pat. No.: 3,342,063 (Smythe et al.); U.S. Pat. No. 3,720,097 (Kron); U.S. Pat. No. 3,999,538 (Philpot, Jr.); U.S. Pat. No. 4,083,363 (Philpot); U.S. Pat. No. 4,149,405 (Ringrose); U.S. Pat. No. 4,165,632 (Weber, et. al.); U.S. Pat. No. 4,517,830 (Gunn, deceased, et. al.); U.S. Pat. No. 4,519,239 (Kiesewetter, et. al.); U.S. Pat. No. 4,554,821 (Kiesewetter, et. al.); U.S. Pat. No. 4,858,127 (Kron, et. al.); U.S. Pat. No. 4,884,577 (Merrill); U.S. Pat. No. 4,947,678 (Hori et al.); U.S. Pat. No. 5,181,415 (Esvan et al.); U.S. Pat. No. 5,257,529 (Taniguchi et al.); U.S. Pat. No. 5,271,398 (Schlain et al.); and U.S. Pat. No. 5,447,440 (Davis, et. al.).

The Smythe '063 patent discloses an apparatus for measuring the viscosity of a blood sample based on the pressure detected in a conduit containing the blood sample. The Kron '097 patent discloses a method and apparatus for determining the blood viscosity using a flowmeter, a pressure source and a pressure transducer. The Philpot '538 patent discloses a method of determining blood viscosity by withdrawing blood from the vein at a constant pressure for a predetermined time period and from the volume of blood withdrawn. The Philpot '363 patent discloses an apparatus for determining blood viscosity using a hollow needle, a means for withdrawing and collecting blood from the vein via the hollow needle, a negative pressure measuring device and a timing device. The Ringrose '405 patent discloses a method for measuring the viscosity of blood by placing a sample of it on a support and directing a beam of light through the sample and then detecting the reflected light while vibrating the support at a given frequency and amplitude. The Weber '632 patent discloses a method and apparatus for determining the fluidity of blood by drawing the blood through a capillary tube measuring cell into a reservoir and then returning the blood back through the tube at a constant flow velocity and with the pressure difference between the ends of the capillary tube being directly related to the blood viscosity. The Gunn '830 patent discloses an apparatus for determining blood viscosity that utilizes a transparent hollow tube, a needle at one end, a plunger at the other end for creating a vacuum to extract a predetermined amount and an apertured weight member that is movable within the tube and is movable by gravity at a rate that is a function of the viscosity of the blood. The Kiesewetter '239 patent discloses an apparatus for determining the flow shear stress of suspensions, principally blood, using a measuring chamber comprised of a passage configuration that simulates the natural microcirculation of capillary passages in a being. The Kiesewetter '821 patent discloses another apparatus for determining the viscosity of fluids, particularly blood, that includes the use of two parallel branches of a flow loop in combination with a flow rate measuring device for measuring the flow in one of the branches for determining the blood viscosity. The Kron '127 patent discloses an apparatus and method for determining blood viscosity of a blood sample over a wide range of shear rates. The Merrill '577 patent discloses an apparatus and method for determining the blood viscosity of a blood sample using a hollow column in fluid communication with a chamber containing a porous bed and means for measuring the blood flow rate within the column. The Hori '678 patent discloses a method for measurement of the viscosity change in blood by disposing a temperature sensor in the blood flow and stimulating the blood so as to cause a viscosity change. The Esvan '415 patent discloses an apparatus that detects the change in viscosity of a blood sample based on the relative slip of a drive element and a driven element, which holds the blood sample, that are rotated. The Taniguchi '529 patent discloses a method and apparatus for determining the viscosity of liquids, e.g., a blood sample, utilizing a pair of vertically-aligned tubes coupled together via fine tubes while using a pressure sensor to measure the change of an internal tube pressure with the passage of time and the change of flow rate of the blood. The Bedingham '328 patent discloses an intravascular blood parameter sensing system that uses a catheter and probe having a plurality of sensors (e.g., an O₂ sensor, CO₂ sensor, etc.) for measuring particular blood parameters in vivo. The Schlain '398 patent discloses a intra-vessel method and apparatus for detecting undesirable wall effect on blood parameter sensors and for moving such sensors to reduce or eliminate the wall effect. The Davis '440 patent discloses an apparatus for conducting a variety of assays that are responsive to a change in the viscosity of a sample fluid, e.g., blood.

Viscosity measuring methods and devices for fluids in general are well-known. See for example, U.S. Pat. No.: 1,810,992 (Dallwitz-Wegner); U.S. Pat. No. 2,343,061 (Irany); U.S. Pat. No. 2,696,734 (Brunstrum et al.); U.S. Pat. No. 2,700,891 (Shafer); U.S. Pat. No. 2,934,944 (Eolkin); U.S. Pat. No. 3,071,961 (Heigl et al.); U.S. Pat. No. 3,116,630 (Piros); U.S. Pat. No. 3,137,161 (Lewis et al.); U.S. Pat. No. 3,138,950 (Welty et al.); U.S. Pat. No. 3,277,694 (Cannon et al.); U.S. Pat. No. 3,286,511 (Harkness); U.S. Pat. No. 3,435,665 (Tzentis); U.S. Pat. No. 3,520,179 (Reed); U.S. Pat. No. 3,604,247 (Gramain et al.); U.S. Pat. No. 3,666,999 (Moreland, Jr. et al.); U.S. Pat. No. 3,680,362 (Geerdes et al.); U.S. Pat. No. 3,699,804 (Gassmann et al.); U.S. Pat. No. 3,713,328 (Aritomi); U.S. Pat. No. 3,782,173 (Van Vessem et al.); U.S. Pat. No. 3,864,962 (Stark et al.); U.S. Pat. No. 3,908,441 (Virloget); U.S. Pat. No. 3,952,577 (Hayes et al.); U.S. Pat. No. 3,990,295 (Renovanz et al.); U.S. Pat. No. 4,149,405 (Ringrose); U.S. Pat. No. 4,302,965 (Johnson et al.); U.S. Pat. No. 4,426,878 (Price et al.); U.S. Pat. No. 4,432,761 (Dawe); U.S. Pat. No. 4,616,503 (Plungis et al.); U.S. Pat. No. 4,637,250 (Irvine, Jr. et al.); U.S. Pat. No. 4,680,957 (Dodd); U.S. Pat. No. 4,680,958 (Ruelle et al.); U.S. Pat. No. 4,750,351 (Ball); U.S. Pat. No. 4,856,322 (Langrick et al.); U.S. Pat. No. 4,899,575 (Chu et al.); U.S. Pat. No. 5,142,899 (Park et al.); U.S. Pat. No. 5,222,497 (Ono); U.S. Pat. No. 5,224,375 (You et al.); U.S. Pat. No. 5,257,529 (Taniguchi et al.); U.S. Pat. No. 5,327,778 (Park); and U.S. Pat. No. 5,365,776 (Lehmann et al.).

The following U.S. patents disclose viscosity or flow measuring devices, or liquid level detecting devices using optical monitoring: U.S. Pat. No. 3,908,441 (Virloget); U.S. Pat. No. 5,099,698 (Kath, et. al.); U.S. Pat. No. 5,333,497. The Virloget '441 patent discloses a device for use in viscometer that detects the level of a liquid in a transparent tube using photodetection. The Kath '698 patent discloses an apparatus for optically scanning a rotameter flow gauge and determining the position of a float therein. U.S. Pat. No. 5,333,497 (Br nd Dag A. et al.) discloses a method and apparatus for continuous measurement of liquid flow velocity of two risers by a charge coupled device (CCD) sensor.

U.S. Pat. No. 5,421,328 (Bedingham) discloses an intravascular blood parameter sensing system.

A statutory invention registration, H93 (Matta et al.) discloses an apparatus and method for measuring elongational viscosity of a test fluid using a movie or video camera to monitor a drop of the fluid under test.

The following publications discuss red blood cell deformability and/or devices used for determining such: Measurement of Human Red Blood Cell Deformability Using a Single Micropore on a Thin Si₃N₄ Film, by Ogura et al, IEEE Transactions on Biomedical Engineering, Vol. 38, No. 8, August 1991; the Pall BPF4 High Efficiency Leukocyte Removal Blood Processing Filter System, Pall Biomedical Products Corporation, 1993.

A device called the “Hevimet 40” has recently been advertised at www.hevimet.freeserve.co.uk. The Hevimet 40 device is stated to be a whole blood and plasma viscometer that tracks the meniscus of a blood sample that falls due to gravity through a capillary. While the Hevimet 40 device may be generally suitable for some whole blood or blood plasma viscosity determinations, it appears to exhibit several significant drawbacks. For example, among other things, the Hevimet 40 device appears to require the use of anti-coagulants. Moreover, this device relies on the assumption that the circulatory characteristics of the blood sample are for a period of 3 hours the same as that for the patient's circulating blood. That assumption may not be completely valid. Also, due to surface alteration, the device requires cleaning after each test.

Notwithstanding the existence of the foregoing technology, a need remains for an apparatus and method for obtaining the viscosity of the blood of a living being in-vivo and over a range of shears and for the provision of such data in a short time span.

All references cited are incorporated herein by reference in their entireties.

### OBJECTS OF THE INVENTION

Accordingly, it is the general object of the present invention to provide an apparatus and methods for meeting that need.

It is a further object of this invention to provide viscosity measuring apparatus and methods for determining the viscosity of circulating blood over a range of shear rates, especially at low shear rates.

It is still yet a further object of this invention to provide an apparatus and methods for determining viscosity of the circulating blood of a living being (e.g., in-vivo blood viscosity measurement) without the need to directly measure pressure, flow and volume.

It is yet another object of this invention to provide an indication of the viscosity of the circulating blood of a living being in a short span of time.

It is yet another object of this invention to provide an indication of the effect of a bioactive agent on the viscosity of the circulating blood of a living being.

It is yet another object of this invention to provide an apparatus and methods for measuring the viscosity of the circulating blood of a living being and with minimal invasiveness.

It is still yet another object of the present invention to provide an apparatus and methods for measuring the viscosity of the circulating blood of a living being that does not require the use of anti-coagulants, or other chemicals or biologically active materials to facilitate measuring.

It is still yet another object of the present invention to provide an apparatus and method for determining the work of the heart of a living being based on the measured viscosity of the circulating blood of the living being.

It is still yet another object of the present invention to provide an apparatus and method for correlating well-known risk factors to a living being by using the viscosity of the circulating blood of the living being over a range of shear rates.

It is still yet another object of the present invention to provide an apparatus and method for detecting the rate of ejection of blood from the heart of a living being based on the pressure pulse of the heart.

It is still yet another object of the present invention to provide a method for explaining the cause of endothelial cell dysfunction of a living being based on hemodynamics.

It is still yet another object of the present invention to provide an apparatus and method for reducing endothelial cell dysfunction in a living being which is caused by oscillating flow of the circulating blood of the living being.

It is still yet another object of the present invention to provide an apparatus and methods for determining the hematocrit of the circulating blood of a living being.

It is still yet another object of the present invention to provide an apparatus and method for determining the plasma viscosity of the circulating blood of a living being.

It is still yet another object of the present invention to provide an apparatus and method for providing high and low blood vessel wall shear stress data.

It is another object of this invention to provide an apparatus and methods for a correlation table that correlates a blood viscosity parameter with a blood pressure parameter to a physician with indicators of high and low blood vessel wall shear stress data.

It is still yet another object of the present invention to provide an apparatus and method for determining the lubricity of the blood of a living being.

It is still yet even another object of the present invention to provide an apparatus and method for detecting the surface tension of the circulating blood of a living being.

It is still yet another object of the present invention to provide an apparatus and method for improving blood perfusion in the lower extremities of a living being.

It is still yet another object of the present invention to provide an apparatus and methods for treating low shear injury through the use of a surface tension analysis means.

It is still yet another object of the present invention to provide apparatus and methods for reducing the work of the heart.

It is moreover another object of the present invention to provide an apparatus and methods for reducing the viscosity of the circulating blood of a living being.

It is even yet another object of this invention to provide an apparatus and methods for determining the coagulation/clotting effects of blood.

It is still yet another object of this invention to provide an apparatus and methods for developing and testing drugs that alter a living being's blood viscosity to achieve Newtonian-type performance at high shear rates.

It is even yet another object of this invention to provide an apparatus and methods for examining the spread of different blood viscosity profiles over a range of shear rates of a living being for diagnostic and treatment purposes.

It is still further another object of this invention to provide prophylactic and therapeutic compositions for controlling at least one property of blood measured by the apparatus and methods of the invention.

It is still further another object of this invention to provide a method for administering a medication to a living being guided by blood parameter information provided by measurement methods and apparatuses of the invention.

It is still yet another object of the present invention to provide a composition pharmaceutically effective to regulate blood viscosity, said composition comprising at least two agents selected from the group consisting of intravenous diluents, red blood cell deformability agents, antiurea agents, oral contraceptives, anti-diabetic agents, antiarrythmics, antihypertensives, antihyperlipidemics, antiplatelet agents, appetite suppressants, antiobesity agents, blood modifiers, smoking deterrent agents, nutritional supplements, endocrine agents, gastrointestinal agents, anti-neoplastic agents, CNS agents, anti-infective agents, anti-asthmatic and pulmonary agents, opthalmic agents, chelating agents and granulocyte colony stimulating factors.

It is still yet another object of the present invention to provide a composition pharmaceutically effective to regulate plasma viscosity, said composition comprising at least two agents selected from the group consisting of anti-diabetics, intravenous solutions, cholesterol-lowering agents, triglyceride-lowering agents, lubricants, homocysteine-reducing agents, and vitamin supplements.

It is still further another object of the present invention to provide a composition pharmaceutically effective to regulate the work of the heart, said composition comprising at least two agents selected from the group consisting of beta-blockers, calcium channel blockers, ACE inhibitors, ACE-II inhibitors, vasodilators, blood pressure reducing agents, viscosity reducing agents and anti-diabetic agents.

It is still yet another object of the present invention to provide a composition pharmaceutically effective to regulate low shear stress, said composition comprising at least two agents selected from the group consisting of beta blockers, calcium channel blockers, ACE inhibitors, ACE-II inhibitors, vasodilators, blood pressure reducing agents, viscosity reducing agents, contractility reducing agents, anti-diabetics, and anti-obesity agents.

It is still yet another object of the present invention to provide a composition pharmaceutically effective to regulate high shear stress, said composition comprising at least two agents selected from the group consisting of intravenous solutions, anti-diabetics, hemodilution agents, anti-platelet agents, lubricity enhancing agents and adhesiveness minimizing agents.

It is still further another object of the present invention to provide a composition pharmaceutically effective to regulate the contractility of the heart, said composition comprising at least two agents selected from the group consisting of beta-blockers, calcium channel blockers, and peripheral antiadrenergic/sympatholytics.

It is still yet another object of the present invention to provide a composition pharmaceutically effective to regulate the thrombogenicity of the heart, said composition comprising at least two agents selected from the group consisting of anti-thrombogenic agents.

It is still yet another object of the present invention to provide a composition pharmaceutically effective to regulate platelet aggregation, said composition comprising at least two agents selected from the group consisting of warfarin, heparin, and anti-platelet agents.

It is still further another object of the present invention to provide a composition pharmaceutically effective to regulate lubricity, said composition comprising at least two agents selected from the group consisting of intravenous fluids, lubricants, anti-adhesives, surfactants, and saponifying agents.

It is still yet another object of the present invention to provide a composition pharmaceutically effective to regulate thixotropy, said composition comprising at least two agents selected from the group consisting of sodium bentonite magma, colloidal clays, colloidal silicon dioxide, and microcrystalline cellulose.

It is still yet another object of the present invention to provide a composition pharmaceutically effective to regulate yield stress, said composition comprising at least two agents selected from the group consisting of gels of colloidal clays, such as sodium bentonite, gels of organic polymers, such as gelatin, agar, pectin, methylcellulose, and high-molecular-weight polyethylene glycol.

It is still further another object of the present invention to provide a composition pharmaceutically effective to regulate endothelial shear injury, said composition comprising at least two agents selected from the group consisting of beta-blockers and viscosity reducing agents.

It is still yet another object of the present invention to provide a composition pharmaceutically effective to regulate coagulability, said composition comprising at least two agents selected from the group consisting of anti-thrombogenics, anti-platelets, heparin, and anti-coagulants.

It is still yet another object of the present invention to provide a composition pharmaceutically effective to regulate coagulation time, said composition comprising at least two agents selected from the group consisting of anti-thrombogenics and anti-platelets, heparin, and anti-coagulants.

It is still further another object of the present invention to provide a composition pharmaceutically effective to regulate agglutination, said composition comprising at least two agents selected from the group consisting of anti-platelets and anti-coagulants.

It is still yet another object of the present invention to provide a composition pharmaceutically effective to regulate clot retraction, said composition comprising at least two agents selected from the group consisting of anti-thrombogenics and anti-platelets, and anti-coagulants.

It is still yet another object of the present invention to provide a composition pharmaceutically effective to regulate clot lysis time, said composition comprising at least two agents selected from the group consisting of anti-thrombogenics, anti-platelets, and anti-coagulants.

It is still further another object of the present invention to provide a composition pharmaceutically effective to regulate prothrombin rates, said composition comprising at least two agents selected from the group consisting of heparin, warfarin and anti-coagulants.

It is still further another object of the present invention to provide a method for adjusting the distribution of a substance through a bloodstream of an organism by altering at least one blood flow parameter of the bloodstream.

It is still further another object of the present invention to provide agents for adjusting the distribution of a substance through a bloodstream of an organism by altering at least one blood flow parameter of the bloodstream, and also to provide compositions comprising such agents and substances.

### SUMMARY OF THE INVENTION

These and other objects of the present invention are achieved by providing a method for distributing and administering a substance through a bloodstream of an organism, said method comprising:

monitoring at least one blood flow parameter of said bloodstream, said at least one blood flow parameter being selected from the group consisting of circulating blood viscosity, absolute viscosity, effective viscosity, low shear viscosity, high shear viscosity, shear rate of circulating blood, work of heart, contractility of heart, thrombogenicity, platelet aggregation, lubricity, red blood cell deformability, thixotropy, yield stress, coagulability, coagulation time, agglutination, clot retraction, clot lysis time, sedimentation rate and prothrombin rate;

administering said substance to said organism such that an amount of said substance enters said bloodstream; and

distributing at least a portion of said amount of said substance to at least one target within said organism,

wherein a distribution parameter of said distributing is adjusted by altering said at least one blood flow parameter.

These and other objects of the present invention are achieved by providing a composition for administration to an organism having a circulatory system, said composition comprising:

a pharmaceutically active agent; and

a distribution agent effective to increase or decrease distribution of said pharmaceutically active agent through said circulatory system by increasing or decreasing at least one blood flow parameter selected from the group consisting of circulating blood viscosity, absolute viscosity, effective viscosity, low shear viscosity, high shear viscosity, shear rate of circulating blood, work of heart, contractility of heart, thrombogenicity, platelet aggregation, lubricity, red blood cell deformability, thixotropy, yield stress, coagulability, coagulation time, agglutination, clot retraction, clot lysis time, sedimentation rate and prothrombin rate,

wherein said distribution agent is not a diluent.

These and other objects of the present invention are achieved by providing a method for determining the work of the heart of a living being based upon the viscosity of the circulating blood of the living being.

These and other objects of the present invention are also achieved by providing a method for determining the rate of ejection of blood from the heart of a living being based on the pressure pulse of the heart.

These and other objects of the present invention are also achieved by providing a method for reducing endothelial cell dysfunction in a living being which is caused by oscillating flow of the circulating blood of the living being. The method comprises the step of reducing the rate of ejection of the blood from the heart of the living being.

These and other objects of the present invention are also achieved by a method for reducing endothelial cell dysfunction in a living being which is caused by oscillating flow of the circulating blood of the living being. The method comprises the step of reducing the viscosity of the circulating blood of the living being.

These and other objects of the present invention are also achieved by a method for reducing endothelial cell dysfunction in a living being which is caused by oscillating flow of the circulating blood of the living being. The method comprises the steps of reducing the rate of ejection of the blood from the heart and reducing the viscosity of the circulating blood of the living being.

These and other objects of the present invention are also achieved by a method for controlling hypertension in a living being. The method comprises the step of administering the combination of β-blocker, ACE inhibitor and blood viscosity reducing drugs together to a living being experiencing hypertension.

These and other objects of the present invention are also achieved by a method for reducing blood viscosity in a living being. The method comprises the step of administering a blood viscosity reducing drug, including but not limited to intravenous diluents, red blood cell deformability agents, antiurea agents, oral contraceptives, anti-diabetic agents, antiarrythmics, antihypertensives, antihyperlipidemics, antiplatelet agents, appetite suppressants, anti-obesity agents, blood modifiers, smoking deterrent agents, nutritional supplements, endocrine agents, gastrointestinal agents, anti-neoplastic agents, CNS agents, anti-infective agents, anti-asthmatic and pulmonary agents, opthalmic agents, chelating agents and granulocyte colony stimulating factors, and any derivatives and/or combinations thereof to a living being.

These and other objects of the present invention are also achieved by an apparatus for determining the hematocrit of the circulating blood of a living being without having to separate red blood cells from the plasma of the circulating blood and wherein the apparatus comprises an optical analysis means.

These and other objects of the present invention are also achieved by an apparatus for determining the viscosity of the plasma of the circulating blood of a living being without the need to centrifuge a portion of the circulating blood of the living being and utilizing single shear rate analysis means.

These and other objects of the present invention are also achieved by a method for estimating blood vessel wall shear stress in high and low shear areas of a blood vessel bifurcation of a living being by correlating a blood viscosity parameter with a blood pressure parameter.

These and other objects of the present invention are also achieved by a method for analyzing the viscosity of the circulating blood of a living being. The method comprises the steps of: (a) determining viscosity data of the living being's circulating blood for a plurality of shear rates over a test run time; (b) segmenting the test run time into a plurality of time segments; and (c) generating a blood viscosity profile for each of the time segments from the beginning of the test run until the end of each of the time segments.

These and other objects of the present invention are also achieved by an apparatus for automatically determining the surface tension of the circulating blood of a living being. The apparatus comprises a blood column height determinator based on capillary rise.

These and other objects of the present invention are also achieved by a method for determining whether a drug reduces or increases the surface tension of the circulating blood of a living being. The method comprising the steps of: (a) determining the surface tension of the circulating blood of a living being utilizing a blood column height determinator based on capillary rise; (b) administering a drug to the living being; and (c) re-determining the surface tension of the circulating blood of the living being utilizing the blood column height determinator to see the change in the surface tension.

These and other objects of the present invention are also achieved by a method for improving blood perfusion to the lower extremities of a living being experiencing peripheral arterial disease. The method comprises the steps of: (a) determining the viscosity of the circulating blood of the living being over a range of shear rates; (b) reducing the viscosity of the circulating blood by administering a substance to the living being or by blood letting; and (c) re-determining the viscosity of the circulating blood of the living being over the range of shear rates to verify the reduction in the viscosity.

These and other objects of the present invention are also achieved by providing an apparatus for determining the deformability of red blood cells of the circulating blood of a living being. The apparatus comprises a plurality of tubes closely adjacent one another and each having an inner diameter different from its neighbor. Furthermore, each of the plurality of tubes has an opening exposed to a flow of circulating blood and each of the tubes being closed at its other end for collecting red blood cells therein.

These and other objects of the present invention are also achieved by an apparatus for detecting the lubricity of the circulating blood of a living being as the blood travels through the vascular system of the living being. The apparatus comprises: a transparent tube for passing a falling column of the circulating blood of the living being; an illuminator for directing light at a portion of the transparent tube that contains a residue left by the falling column; a detector for detecting any light that passes through the transparent tube and residue and generating corresponding detection data; and calculation means for receiving the detection data and generating a lubricity value based on the detection data.

These and other objects of the present invention are also achieved by prophylactic and therapeutic compositions and methods for controlling at least one property of blood measured by the apparatus and methods of the invention.

These and other objects of the present invention are also achieved by a method for administering a medication to a living being, said method comprising: (a) providing an apparatus according to the invention, which is adapted to measure at least one blood flow parameter of the living being selected from the group consisting of circulating blood viscosity, absolute viscosity, effective viscosity, low shear viscosity, high shear viscosity, shear rate of circulating blood, work of heart, contractility of heart, thrombogenicity, platelet aggregation, lubricity, red blood cell deformability, thixotropy, yield stress, coagulability, coagulation time, agglutination, clot retraction, clot lysis time, sedimentation rate and prothrombin rate; (b) supplying a sample of the living being's blood to the at least one apparatus; and (c) measuring the at least one blood flow parameter to determine whether and how to administer the medication to the living being, wherein the apparatus is at least one member selected from the group consisting of a circulating blood viscometer, an electronic hematocrit analyzer, a plasma viscosity analyzer, a blood lubricity detector, a red blood cell deformability analyzer and a surface tension analyzer.

### DESCRIPTION OF THE DRAWINGS

Other objects and many of the intended advantages of this invention will be readily appreciated when the same becomes better understood by reference to the following detailed description when considered in connection with the accompanying drawings wherein:

FIG. 1 is a block diagram of a dual riser/single capillary (DRSC) viscometer;

FIG. 1A is a functional diagram of the first embodiment of the DRSC viscometer during the viscosity test run;

FIG. 2 is a block diagram of another DRSC viscometer;

FIG. 2A is a functional diagram of the second embodiment of the DRSC viscometer during the viscosity test run;

FIG. 3A is the graphical depiction of the cardiac output of the heart of a living being;

FIG. 3B is a graphical depiction of the pressure pulse of the heart of a living being;

FIG. 3C is a blood viscosity vs. time plot for a living being;

FIG. 3D is a graphical depiction of the pressure pulse of the heart of a living being having a first contractility, and another pressure pulse of the heart having a second increased contractility;

FIG. 3E is a graphical depiction of how the contractility of the heart of a living being can be determined from the pressure pulse curve;

FIG. 4 is a flow diagram of a portion of an artery showing a bifurcation;

FIG. 5A is an enlarged view of healthy, normal endothelial cells located along a portion of an arterial wall;

FIG. 5B is an enlarged view of dysfunctional endothelial cells, e.g., endothelial cells located along a portion of an arterial wall opposite a bifurcation;

FIG. 6 is a functional diagram of a hematocrit analyzer of the present invention;

FIG. 7 is an enlarged view of a portion of the hematocrit analyzer showing a predetermined window used in the hematocrit analysis;

FIG. 8 is an alternative lumen for use in the hematocrit analyzer;

FIGS. 9A-9C together constitute the plasma viscosity analyzer;

FIG. 10 depicts a graphical representation of the respective columns of fluid in the riser tubes of either the first or second embodiment of the DRSC viscometer during the viscosity test run;

FIG. 11 depicts a graphical representation of the absolute viscosity profile versus the effective viscosity profile corresponding to FIG. 10;

FIG. 12A depicts a typical graphical representation of the absolute viscosity profile versus the effective viscosity profile for a living being;

FIG. 12B depicts a graphical representation of the absolute viscosity profile versus the effective viscosity profile for a healthy living being;

FIG. 12C depicts a graphical representation of the effective viscosity profile for a living being under test versus the effective viscosity profile of a normal, healthy individual;

FIG. 13 is a table for presenting blood pressure and blood viscosity parameters in a matrix fashion for indicating both high and low blood vessel wall shear stress data;

FIG. 14A is an enlarged view of the top of the riser having a falling blood column showing a meniscus;

FIG. 14B depicts a blood lubricity detector used in conjunction with the riser tube of FIG. 14A;

FIG. 14C depicts blood lubricity plots for several living beings under test;

FIG. 15 depicts a red blood cell deformability analyzer;

FIGS. 16A-16B depict a surface tension analyzer;

FIG. 17 depicts a graphical representation of the respective columns of fluid in the riser tubes of either the first or second embodiment of the DRSC viscometer during the viscosity test run wherein the height vs. time data is segmented into a plurality of shear rate regions;

FIGS. 18A and 18B are blood viscosity profiles for a patient A and a patient B, respectively, based on the various shear rate regions depicted in FIG. 17;

FIG. 19 depicts one full blood viscosity profile including the extreme high and low shear rate ranges;

FIG. 20 depicts a method for improving blood profusion in the lower extremities of a living being;

FIG. 21 depicts a method for treating low shear injury through the use of a surface tension analyzer; and

FIG. 22 depicts red blood cell bonding at both a high shear and low shear conditions.

### DETAILED DESCRIPTION OF THE PREFERRED EMBODIMENTS

As stated previously, the present application is a Continuation-in-Part of the copending U.S. application filed Apr. 9, 2001 entitled IN VIVO DELIVERY METHODS AND COMPOSITIONS and having the identifying Attorney Docket No. V1025/20099, which application in turn is a Continuation-in-Part of the copending U.S. application Ser. No. 09/819,924 filed Mar. 28, 2001 entitled IN VIVO DELIVERY METHODS AND COMPOSITIONS, which application in turn is a Continuation-in-Part of a co-pending U.S. patent application Ser. No. 09/628,401, filed Aug. 1, 2000, entitled APPARATUS & METHODS FOR COMPREHENSIVE BLOOD ANALYSIS, INCLUDING WORK OF, AND CONTRACTILITY OF, HEART, AND THERAPEUTIC APPLICATIONS AND COMPOSITIONS THEREOF, which in turn is a Continuation-in-Part of Co-Pending application Ser. No. 09/501,856, filed Feb. 10, 2000, entitled METHOD OF ANALYZING DATA FROM A CIRCULATING BLOOD VISCOMETER FOR DETERMINING ABSOLUTE AND EFFECTIVE BLOOD VISCOSITY, which in turn is a Continuation-in-Part of Co-Pending application Ser. No. 09/439,795, filed Nov. 12, 1999, entitled DUAL RISER/SINGLE CAPILLARY VISCOMETER, both of which are assigned to the same Assignee as the present invention and whose entire disclosures are incorporated by reference herein. The apparatus disclosed in application Ser. No. 09/439,795 provides the medical community the ability to observe the instantaneous circulating blood viscosity characteristic that has, up until now, not been detectable by conventional blood viscometers.

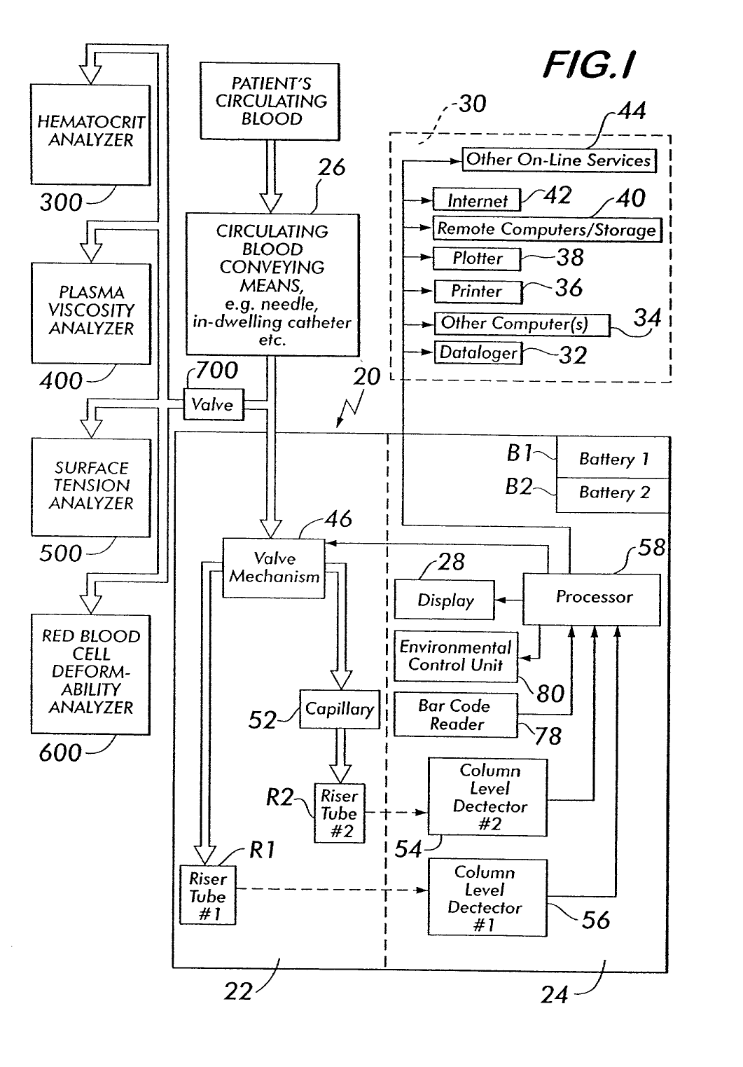

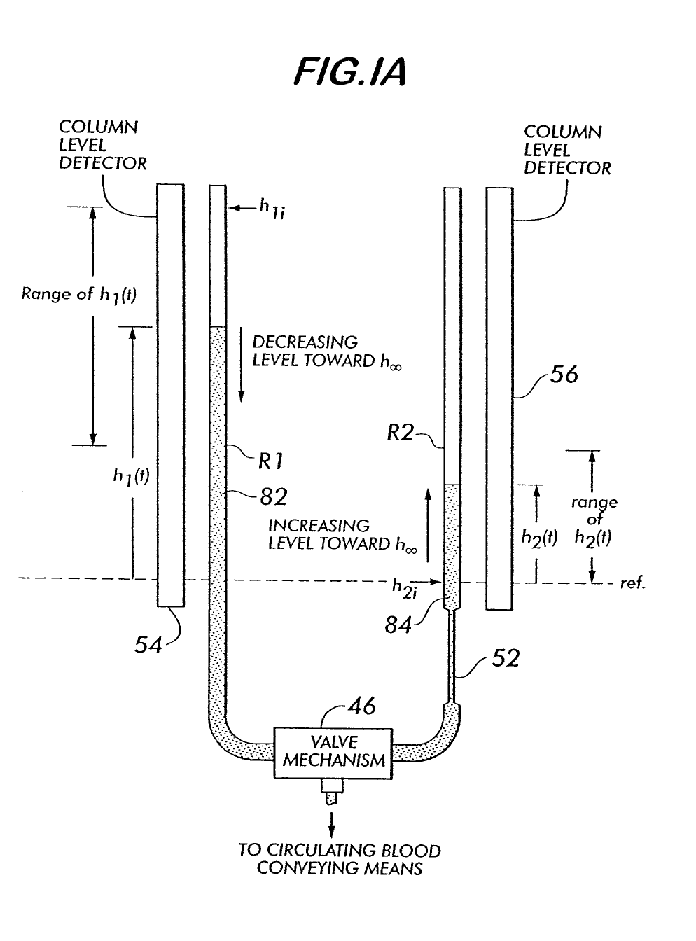

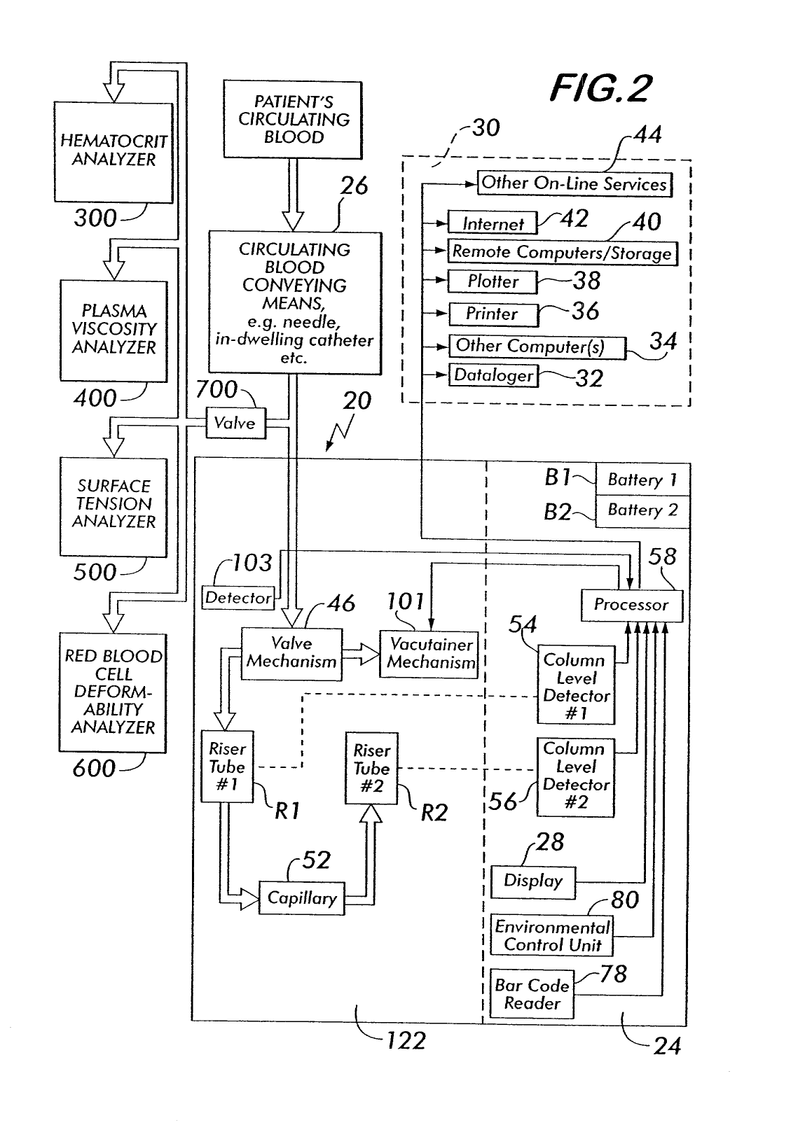

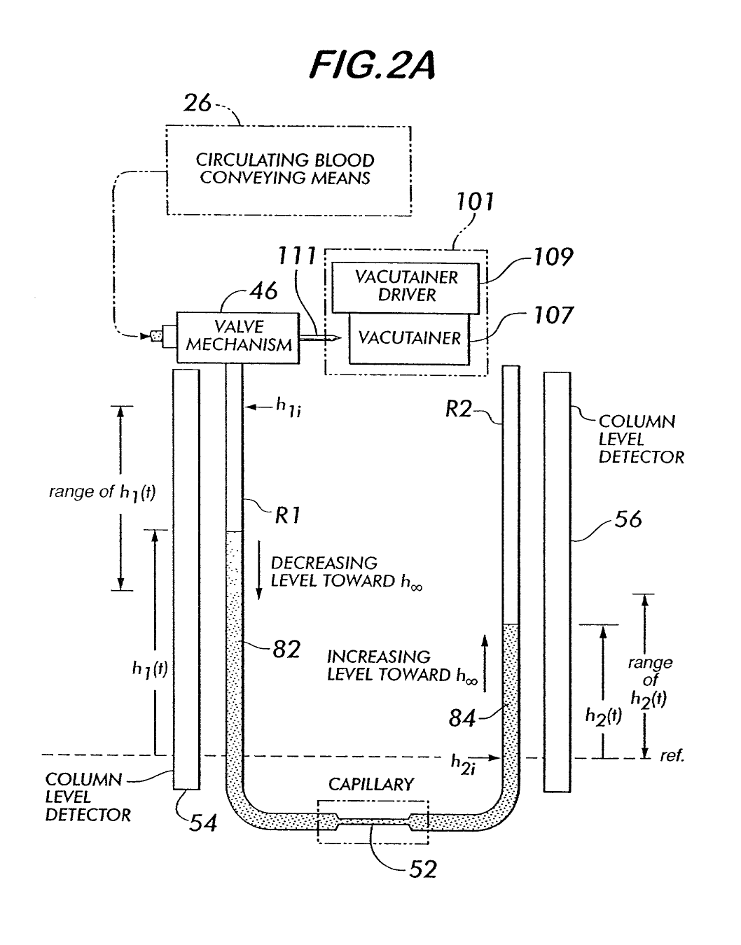

In particular, the apparatus disclosed in application Ser. No. 09/439,795 comprises a first embodiment of a dual riser/single capillary (DRSC) viscometer shown in FIGS. 1 and 1A, and a second embodiment of the DRSC viscometer shown in FIGS. 2 and 2A, each of which measures the viscosity of circulating blood, including whole blood, of a living being. For purposes of the present invention, either embodiment can be used to achieve the method described herein.

Basically, the DRSC viscometers 20 (FIG. 1) and 120 (FIG. 2) comprise a blood receiving means 22 and 122, respectively, and an analyzer/output portion 24. The patient is coupled to the DRSC viscometers 20/120 through a circulating blood conveying means 26, e.g., a needle, an IV needle, an in-dwelling catheter, etc., or any equivalent structure that can convey circulating blood from a patient to the DRSC viscometers 20/120. The analyzer/output portion 24 includes a microprocessor 58 that, among other things, calculates the circulating blood viscosity based on the information that it receives from the blood receiving means 22/122. A display 28 is also provided for presenting the viscosity information, as well as other information to the operator. The analyzer/output portion 24 may also provide this information to other suitable output means 30, such as a datalogger 32, other computer(s) 34, a printer 36, a plotter 38, remote computers/storage 40, to the Internet 42 or to other on-line services 44.

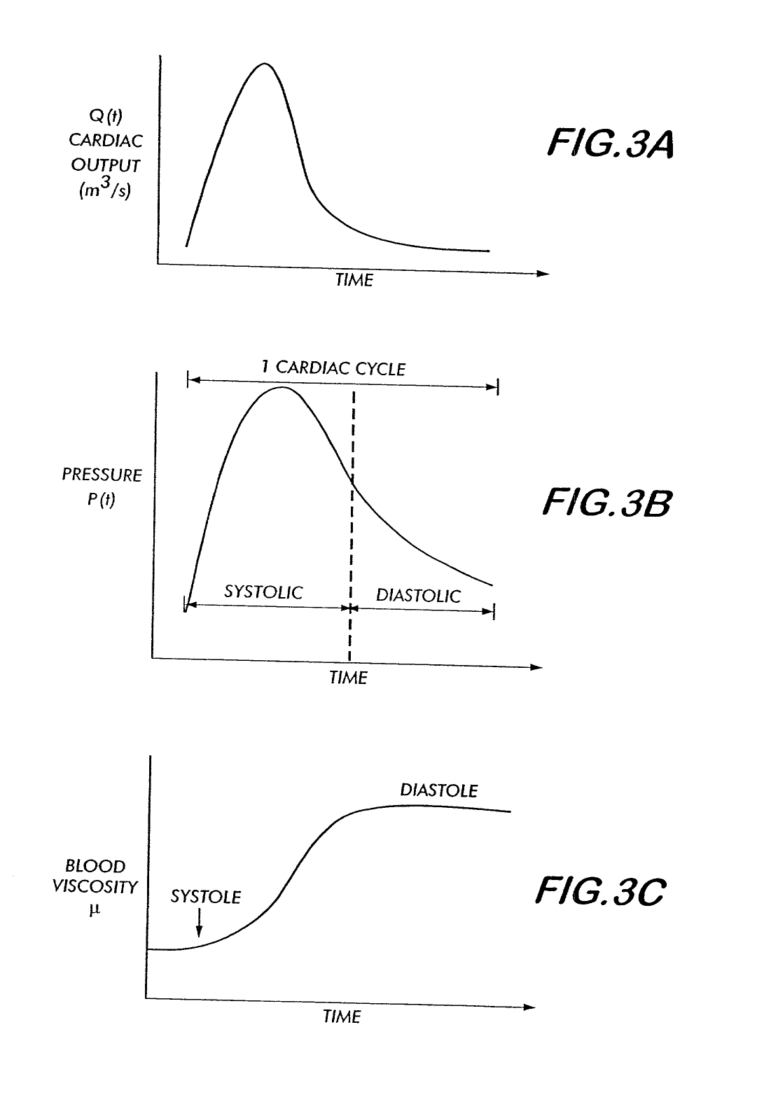

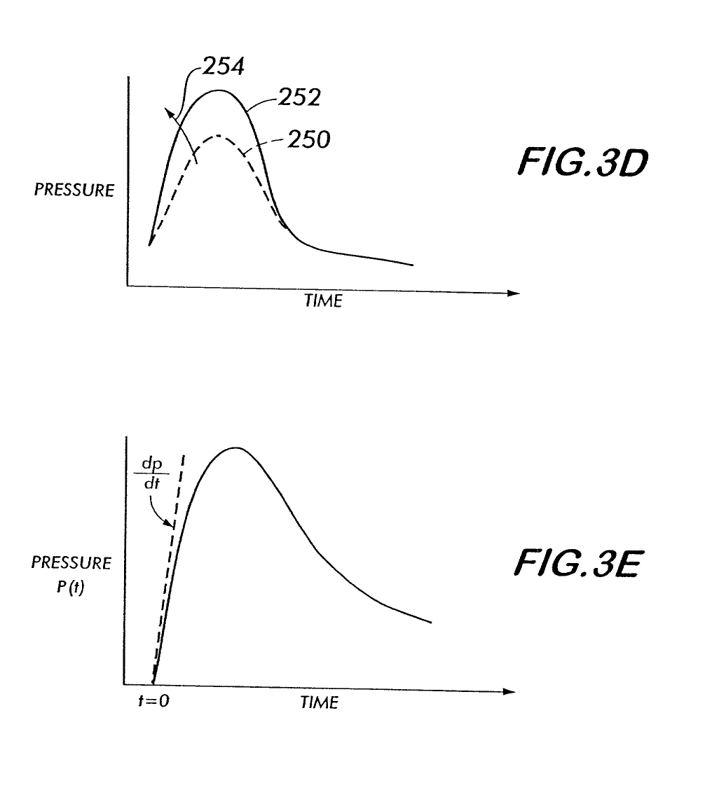

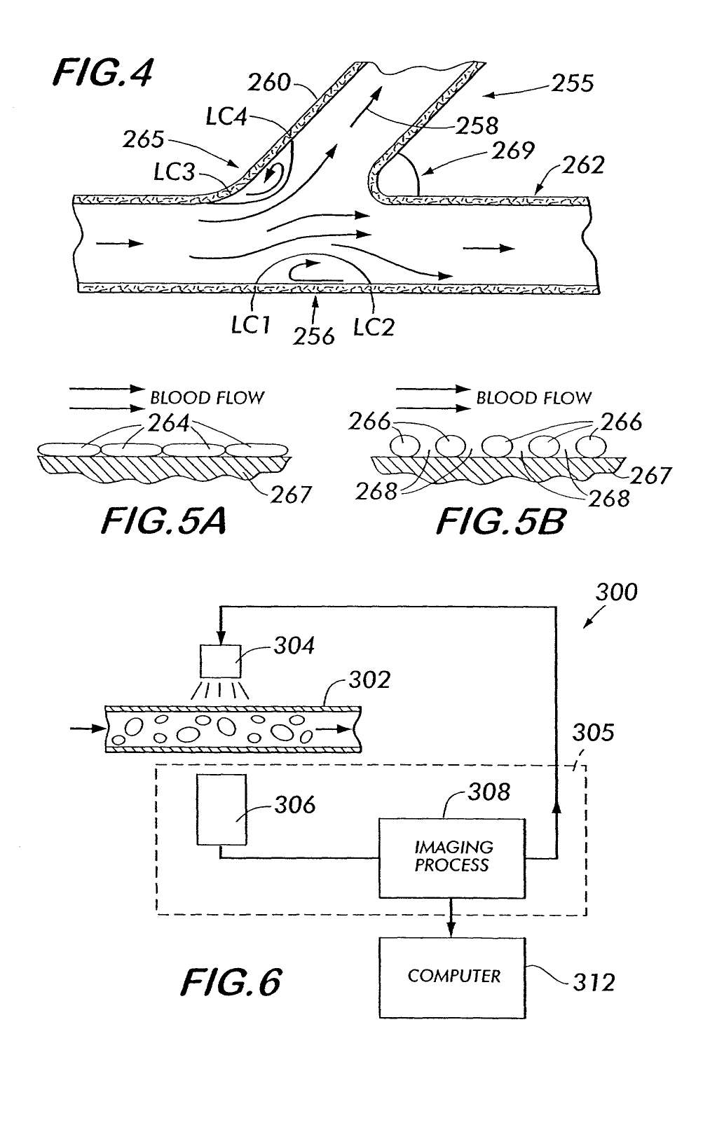

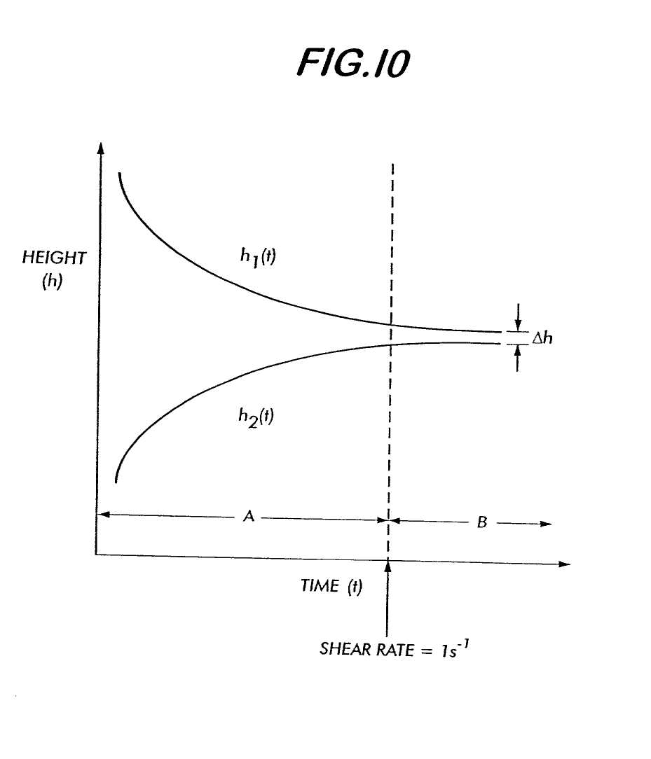

The blood receiving means 22/122 basically comprises a valve mechanism 46 coupled between a first riser tube R1 and a second riser tube R2 (FIGS. 1-2), or coupled to one end of one of the riser tubes (FIGS. 3-4), for controlling the input circulating blood flow into the DRSC viscometers 20/120. In addition, a capillary tube 52 of known dimensions is coupled to one of the riser tubes (e.g., as shown in FIG. 2), or is coupled between the riser tubes (e.g., as shown in FIG. 4). In general, the valve mechanism 46 in both embodiments establishes a first initial position, h₁ᵢ, of a column of blood (h₁) in one of the riser tubes (e.g., R1) and a second initial position, h₂ᵢ, of another column of blood (h₂) in the other of the riser tubes (e.g., R2). The valve mechanism 46 then isolates these columns of blood from the input circulating blood flow, resulting in the oppositely-moving columns of blood away from their initial positions as shown in FIGS. 1A and 2A. Just prior to this isolation and during the movement of the columns of blood, each column of blood is monitored by a respective column level detector 54 and 56 which send their data to the microprocessor 58. As a result, the column level detectors 54/56 collect data (h₁(t) and h₂(t)) regarding the movement of these respective columns of blood, which can also be plotted (FIG. 10) and then displayed on the display screen 28.

It should be understood that it is within the broadest scope of the invention to replace one of the two column level detectors 54/56 with a single point detector in either of the two viscometers 20 and 120 (FIGS. 1/1A and FIGS. 2/2A) as disclosed in application Ser. No. 09/573,267, filed on May 18, 2000, entitled DUAL RISER/SINGLE CAPILLARY VISCOMETER and whose entire disclosure is incorporated by reference herein. This modification is based on the symmetry of the column of blood height (i.e., h₁(t) and h₂(t)) vs. time data (see FIG. 10). As long as one of the two columns of blood 82/84 is monitored, the height vs. time data for the other column of blood can be generated by using a single height point from that column. In the invention of the present application, it is only necessary to monitor the change in position of one of the columns of blood in either riser tube R1 or riser tube R2 and to detect only one point from the other column of blood. The preferred method/means is to monitor the rising column of blood 84 which occurs in riser tube R2 and to detect the initial viscosity test run level (i.e., h₁ᵢ, as discussed in application Ser. No. 09/439,795) of the column of blood 82 in riser tube R1. Thus, it is within the broadest scope of this invention to cover a monitor that monitors either one of the moving columns of blood (which also includes methodologies known in the art such as monitoring the change in position, column height, weight, volume, mass, etc.) but not both columns (as is disclosed in application Ser. No. 09/439,795) and a single point detector for detecting one point from the other moving column of blood.

Where both column level detectors 54/56 are used, or just one column level detector is used, a blood column movement indicator is also provided. This indicator provides either a visual and/or audible indication of the blood column movement. For example, as either the falling column moves downward or the rising column moves upward, the indicator provides a flashing light whose flash rate is proportional to the speed of either the falling or rising column movement. Alternatively, or in addition, the indicator provides a continuous beeping sound whose beeping rate is proportional to the speed of either the falling or rising column. As a result, when the viscosity test run begins, and the falling and rising columns are moving at high rates, the indicator flashes and/or beeps at a high rate; near the end of the viscosity test run when the falling and rising columns are moving very slowly, the indicator flashes very slowly and/or beeps a slow rate. One example of the blood column movement indicator comprises including a sound card (e.g., Sound Blaster AWE64 manufactured by Creative) and speaker (not shown) in the display 28. As the columns of blood rise or fall, the processor 58 activates the speaker card which causes the speaker to emit a sound whose intensity varies with the speed of the blood column. In addition, the graphical depiction of the two height vs. time plots in the graphical display 61 can flash at a rate that varies with the speed of the column movement.

Based on the above discussion, the apparatus and method of the present invention are now discussed; the details of the other components in the blood receiving means 22/122 depicted in FIGS. 1-2A are discussed in application Ser. No. 09/439,795 and application Ser. No. 09/501,856 and are not repeated here. Suffice it to say that using either one of the embodiments 20/120 the viscosity (μ) of the circulating blood of the living being can be determined as well as the absolute viscosity and effective viscosity profiles for that living being.

As shown in FIGS. 1 and 2, several additional analyzers have been added in tandem with the circulating blood viscosity determination. These additional analysis means basically take advantage of the single intubation of the living being by the circulating blood conveying means 26. In particular, a hematocrit analyzer 300, plasma viscosity analyzer 400, surface tension analyzer 500, and red blood cell deformability analyzer 600. The details of each of these will be discussed in below. Furthermore, to avoid overflowing these analyzers 300-600 with circulating blood, a valve 700 is used which permits a predetermined amount of blood to enter the respective analyzer and then closes off the path to these means.

Because the viscosity μ(t) of the circulating blood of the living being can be determined (as set forth in application Ser. No. 09/439,795 and application Ser. No. 09/501,856) as well as the absolute viscosity and effective viscosity profiles (as set forth in application Ser. No. 09/501,856) for that living being, certain parameters of the heart can also now be determined: work of the heart (WOH) and contractility of the heart (CON).

WOH can be estimated from the following equation:

$$Work of the Heart ( WOH ) = \frac{1}{T} {\int }_{0}^{T} P ( t ) \cdot Q ( t ) ⅆ t$$

where:

P(t) is the pressure pulse curve of the heart (FIG. 3B);

Q(t) is the cardiac output (see FIG. 3A); and

T represents the period of one cardiac cycle.

In any flow system, the flow resistance comes from the piping arrangement and the type of fluids. As blood viscosity increases, the flow rate (i.e., cardiac output) decreases if the size of pump remains constant. In a steady state flow, the Poiseuille flow describes the flow rate (Q) in terms of the viscosity (μ) of the fluid, the length (L) of the tube, the inside diameter (d) of the tube and the pressure drop (ΔP) across the length of the tube, and is given as:

$$Q = \frac{π {d}^{4} Δ P}{128 µ L} \qquad ( Laminar flow )$$

The pumping power to generate Q can be given as pumping power=Q·ΔP.

For a pulsatile blood flow, the WOH is given as:

$$\frac{1}{T} {\int }_{0}^{T} P ( t ) \cdot Q ( t ) ⅆ t$$

where, for any given instantaneous flow,

$$Q ( t ) = \frac{π {d}^{4} Δ P ( t )}{128 µ L}$$

As used in the present context with regard to a living being's vascular system, the term ΔP(t) represents the pressure difference between the ends of a blood vessel of a fixed diameter and length. The vascular system between the heart (aorta) and vein is composed of blood vessels having different diameters and corresponding lengths which are known in the art. Since the pressure at the capillary bed can be approximated to be zero, the term, ΔP(t), can be approximated with the pressure pulse term, P(t), such that the equation for WOH is defined as:

$$WOH = \frac{1}{T} \cdot \frac{π {d}^{4}}{138 L} {\int }_{0}^{T} P ( t ) \cdot [ \frac{P ( t )}{μ ( t )} ] ⅆ t = \frac{π {d}^{4}}{128 TL} {\int }_{0}^{T} [ \frac{{P}^{2} ( t )}{μ ( t )} ] ⅆ t$$

where “d” and “L” represent average diameter and length of the entire vascular system of a living being. The pressure pulse of the heart, P(t), can be detected by conventional medical equipment using, e.g., skin sensors and a digital storage oscilloscope. Thus, because the viscosity μ(t) of the circulating blood of a living being can be determined (FIG. 3C) using the viscometers 20/120, it is now possible to determine the WOH of the living being.

The contractility of the heart (COH) is the rate of ejection of blood by the left ventricle of the heart (FIG. 3D). The faster the heart squeezes blood out of the left ventricle, the greater is the contractility of the heart. In particular, as the contractility of the heart increases (from the dotted line 250 which indicates a first lower COH to the solid line 252 which indicates a second higher COH in the direction of the arrow 254), the pressure pulse wave becomes steeper during systole. Another term for COH is the “pulsatility” of the heart.

Quantitatively, the contractility can be measured from the pressure pulse curve (FIG. 3E). The slope of the pressure pulse curve in the beginning of systole represents how fast the left ventricle of the heart ejects blood. Hence, the slope represents the contractility of the heart. Mathematically,

$$COH = slope = {( \frac{ⅆ p}{ⅆ t} )}_{@ t = 0}$$

The importance of COH is discussed next with respect to blood viscosity and blood vessel wall shear stress.

Arterial disease often occurs at bifurcations (FIG. 4), but not in straight vessels. Hence, it is often called site-specific disease. In particular, it is known that blood flow recirculation occurs on the wall 256 opposite the flow divider 255 but has heretofore not been explained. One of the reasons may be hemodynamics.

In a bifurcation (FIG. 4), there is a flow 258 to the branch vessel 260. Thus, because the mass in the main vessel 262 decreases, the pressure at location LC2 increases compared to the pressure at location LC1, resulting in P_{LC2}>P_{LC1}, where “P” stands for pressure. This pressure differential forces some fluid elements to move upstream, producing a recirculation flow. A recirculation flow in an unsteady, pulsatile blood flow means that the wall shear stress between LC1 and LC2 is alternating between a negative value (e.g., −5 dyne/cm²) and a positive value (i.e., +5 dyne/cm²). This same type of pressure differential also occurs at the proximal side 265 of the flow divider 255. In particular, a recirculation flow in an unsteady, pulsatile blood flow also occurs between location LC3 and location LC4 wherein P_{LC4}>P_{LC3}. This pressure differential forces some fluid elements to move upstream, producing a recirculation flow in the branch flow 258. This means that the wall shear stress between LC3 and LC4 is alternating between a negative value (e.g., −5 dyne/cm²) and a positive value (i.e., +5 dyne/cm²). This alternating wall shear stress can be viewed as a sandpaper or abrading effect. The effect of this alternating shear on endothelial cells is very serious and the key to the arterial disease. In particular, endothelial cells 266 (FIG. 5B) become more rounded, forming dysfunctional endothelial cells which expose leaky sites, whereas normal, healthy endothelial cells 264 are elongated and contiguous (FIG. 5A).

As shown in FIG. 5B, endothelial cells (hereinafter, “E-cells”) in a recirculating area become more rounded than elongated along the flow direction. Rounded E-cells are more permeable so that lipid and other macromolecules can move through the endothelial cell layer from blood to arterial wall 267 via the gaps, i.e., leaky sites 268. Hence, the E-cells do not perform their normal function and are called dysfunctional E-cells. When E-cells become rounded, the life of the cells become short, i.e., the cell turnover becomes high. When E-cells become dysfunctional, this causes a series of biological responses, including the production of nitric oxide (NO). In short, E-cells become dysfunctional due to oscillating/alternating wall shear stress in the low shear zones (1) at the wall 256 opposite the flow divider 255 and (2) at the proximal side 265 of the branch vessel 255.

By reducing the COH of the heart, one can reduce the magnitude of the oscillating wall shear stress in low-shear zones. It should be noted that it is not desirable to “reduce” low shear stress. What is desirable is to reduce the +/− swing. When a living being has a high contractility, the wall shear stress at the opposite wall 256 may vary from −10 to +10 dyne/cm². By administering a drug to reduce the contractility, one can correct the wall shear stress swinging from −3 to +3, wherein the E-cells will be much less dysfunctional. Thus, by reducing the contractility of the heart one can normalize the E-cell, reduce the number of dysfunctional E-cells, reduce cell turnover, reduce leaky sites, and reduce permeability of E-cells. The benefit of reducing contractility is to reduce the transport of lipids and other macromolecules across the E-cell layer, thus preventing the initiation and progression of arterial disease or atherosclerosis.

There are a number of drugs (such as beta-blockers) that can reduce the contractility of the heart. Smoking is known to increase the contractility of the heart, thus accelerating the progress of atherosclerosis. Alcohol is well-known to relax the muscle of the left ventricle of the heart, thus decreasing the contractility of the heart. Caffeine (coffee) can increase the contractility of the heart. Thus, well-known risk factors, such as those addressed above, can be correlated to the contractility of the heart of a living being.

There is also a relationship between blood viscosity (μ) and dysfunctional E-cells. Blood viscosity affects the global hemodynamics at arterial vessels, particularly at arterial bifurcation 255, thus affecting E-cells. As blood viscosity increases, the flow separation zone increases and the magnitude of the alternating wall shear stress (i.e., the positive and negative values) is amplified. As blood viscosity decreases, the magnitude (or level) of the alternating wall shear stress decreases, resulting in more healthy E-cells, i.e., less dysfunctional E-cells. The shape of the E-cells is less round and the E-cell turnover decreases. Hence, the reduced blood viscosity can reduce the transport of lipids and macromolecules across the E-cell layer (i.e., intima). Therefore, any drug compositions reducing blood viscosity can reduce the number of dysfunctional E-cells which are often called intimal injury at the early stage of atherosclerosis. For example, drugs known to reduce viscosity include, but are not limited to, intravenous diluents, red blood cell deformability agents, antiurea agents, oral contraceptives, anti-diabetic agents, antiarrythmics, antihypertensives, antihyperlipidemics, antiplatelet agents, appetite suppressants, antiobesity agents, blood modifiers, smoking deterrent agents, nutritional supplements and any derivatives and/or combinations thereof. Preferably, oral contraceptives, antiplatelet agents and antihyperlipidemics. More preferable, aspirin and its derivatives and any pharmaceutical compound combined with aspirin, oral contraceptives consisting essentially of levonorgestrel, estrogen, progestin, estradiol, ethinyl estradiol, ethynodiol, medroxyprogesterone, desogestrel, cyproterone, norethindrone, gestodene, norgestrel, mestranol, or norgestimate, including their salts, derivatives and any combinations thereof, antihyperlipidemic agents, and abciximab which is commercially available from Eli Lilly & Co. as the prescription product ReoPro®.

Suitable intravenous diluents include, but are not limited to, saline, deionized water, and any derivatives and/or combinations thereof.

Suitable antidiabetic agents include, but are not limited to, metformin, acarbose, insulins including all salts and crystalline forms, chlorpropamide, glipizide, glyburide, tolazamide, glimepiride, troglitazone, pioglitazone, repaglinide, losartan potassium, candesartan cilexetil, irbesartan, mitiglinide, trendolapril/verapamil, nateglinide, repaglinide, and any derivatives and/or combinations thereof.

Suitable antihypertensive agents include, but are not limited to, nifedipine, nisoldipine, nicardipine, bepridil, isradipine, nimodipine, felodipine, amlodipine, diltiazem, verapamil, isosorbide mononitrate, isosorbide dinitrate, nitroglycerin, hydralazine, minoxidil, hydrochlorothiazide, chlorothiazide, indapamide, metolazone, furosemide, bumetanide, ethacrynic acid, torsemide, spironolactone, triamterene, acetazolamide, mannitol, atenolol, bisoprolol, pindolol, metoprolol, timolol, nadolol, propanolol, carvedilol, captopril, fosinopril, benazepril, lisinopril, enalapril, quinapril, losartan, valsartan, irbesartan, eprosartan, trandolapril, fenoldopam, ramipril, doxazosin, milrinone, benidipine, lemakalim, fantofarone, lemildipine, pirmenol, clentiazem, nebivolol, oxodipine, sematilide, pranidipine, nifekalant, aranidipine, barnidipine, lacidipine, bucindolol, azelnidipine, dofetilide, losartan potassium, eprosartan, ibutilide, candesartan, watanidipine, irbesartan, lercanidipine, landiolol, telmisartan, furnidipine, valsartan, azimilide, carvedilol, CHF 1521, trandolapril/verapamil, losartan, valsartan/hydrochlorothlazide, enalapril/nitrondipine, sotalol, arbutamine, olmesartan, conivaptan, and any derivatives and/or combinations thereof.

Suitable antihyperlipidemic agents include, but are not limited to, lovastatin, atorvastatin, cerivastatin, simvastatin, fluvastatin, cholestyramine, colestipol, clofibrate, gemfibrozil, fenofibrate, pamaqueside, pitavastatin, and any derivatives and/or combinations thereof.

Suitable appetite suppressants and anti-obesity agents include, but are not limited to, phentermine, phendimetrazine, sibutramine, orlistat and any derivatives and/or combinations thereof.

Suitable blood modifiers include, but are not limited to, aspirin, warfarin, enoxaparin, heparin, low molecular weight heparin, cilostazol, clopidogrel, ticlopidine, tirofiban, abciximab, dipyridamole, plasma protein fraction, human albumin, low molecular weight dextran, hetastarch, reteplase, alteplase, streptokinase, urokinase, dalteparin, filgrastin, immunoglogulin, ginkolide B, clopidogrel, hirudins, foropafant, rocepafant, bivalirudin, dermatan sulfate mediolanum, eptilibatide, tirofiban, thrombomodulin, abcxmab, low molecular weight dermatan sulfate-opocrin, eptacog alfa, argatroban, fondaparinux sodium, tifacogin, lepirudin, desirudin, OP2000, melagatran, roxifiban, parnaparin sodium, human hemoglobin (Hemosol), bovine hemoglobin (Biopure), human hemoglobin (Northfield), antithrombin III, RSR13, heparin-oral (Emisphere) transgenic antithrombin III, H37695, enoxaparin sodium, mesoglycan, CTC111, bivalirudin, and any derivatives and/or combinations thereof.

Additionally, distribution agents and blood modifiers can be selected from the following list, and titrated for optimal concentrations based on the information and techniques described elsewhere herein: acacia; acacia mucilage; acetic acid; acetic acid, glacial; acetic anhydride; acetone sodium bisulfite; acetyl tributyl citrate; acetylated monoglycerides; acetylcysteine; acrylates copolymer; adcote 72A103; aerosil 380; aerosil-200; aerotex resin 3730; air; albumin aggregated; albumin colloidal; albumin human; alcohol (especially ethanol); alcohol, dehydrated; alcohol, denatured; alcohol, diluted; alginic acid; alkyl ammonium sulfonic acid betaine; alkyl aryl sodium sulfonate; allantoin; althea; aluminum acetate; aluminum hydroxide; aluminum hydroxide-sucrose, hydrated; aluminum hydroxide gel; aluminum hydroxide gel F 500; aluminum hydroxide gel F 5000; aluminum hydroxide gel, dried; aluminum oxide; aluminum polyester; aluminum potassium sulfate; aluminum silicate; aluminum starch octenylsuccinate; aluminum stearate; aluminum sulfate; alzamer-50; amberlite; amerchol L101; amerchol-CAB; ammonia; ammonia solution; ammonium acetate; ammonium calcium alginate; ammonium chloride; ammonium hydroxide; ammonium phosphate, dibasic; ammonium salt of C-12-C-15 linear primary alcohol ethoxylate; ammonium sulfate; ammonyx; amphoteric-2; amphoteric-6; anethole; anidrisorb 85/70; anise extract; anise oil; anise, Star; anoxid SBN; antifoam; antifoam DC; antipyrine; Aquacoat; Aquacoat ECD; aquaphor; arginine; arlatone 289; ascorbic acid; ascorbyl palmitate; aspartame; aspartic acid; balsam canada; balsam, fir; barium sulfate; beeswax; beeswax, synthetic, bentonite; benzaldehyde; benzalkonium chloride; benzenesulfonic acid; benzethonium chloride; benzododecinium bromide; benzoic acid; benzoin; benzyl alcohol; benzyl alcohol; benzyl benzoate; benzyl chloride; beta-naphthol; boric acid; buffer, acetic acid-sodium acetate; buffer, citric acid-sodium citrate; butane; butyl alcohol, tertiary; butylated hydroxyanisole; butylated hydroxytoluene; butylene glycol; butylparaben; caffeine; calcium; calcium acetate; calcium ascorbate; calcium carbonate; calcium carbonate, precipitated; calcium chloride; calcium gluceptate; calcium hydroxide; calcium lactate; calcium phosphate; calcium phosphate dibasic dihydrate; calcium phosphate, dibasic; calcium phosphate, dibasic, dihydrate; calcium phosphate, tribasic; calcium pyrophosphate; calcium silicate; calcium stearate; calcium sulfate; calcium sulfate dihydrate; calcium sulfate, anhydrous; caldiamide sodium; calteridol calcium; candelilla wax, canola oil; caprylic/capric diglyceryl succinate; caprylic/capric triglyceride; caramel; carbomer; carbomer 1342; carbomer 934; carbomer 934P; carbomer 940; carbomer 941; carbomer 974; carbon dioxide; carboxy vinyl copolymer; carboxymethyl starch; carboxymethylamylopectin sodium; carboxymethylcellulose; carboxymethylcellulose calcium; carboxymethylcellulose sodium; carboxypolymethylene; cardamom; carmine; carmine solution; carminic acid; carnauba wax; carnauba yellow wax; carrageenan; carrageenan salt; cassia oil; castor oil; castor oil hydrogenated; cellulose; cellulose acetate; cellulose acetate phthalate; cellulose microcrystalline/carboxymethylcellulose sodium; cellulose microcrystalline, aqueous; cellulose, microcrystalline; cellulose, oxidized; cellulosic polymers; ceresin; ceteareth-12; ceteareth-15; ceteareth-20; ceteareth-30; cetearyl alcohol; cetearyl octanoate; ceteth-10; ceteth-2; ceteth-20; cetyl alcohol; cetyl esters; cetyl palmitate; cetylpyridinium chloride; cherry; cherry juice; chlorobutanol; chlorobutanol hemihydrate; chlorobutanol, anhydrous; chlorocresol; chloroxylenol; cholesterol; choleth; cinnamaldehyde; cinnamon; cinnamon oil; citric acid; citric acid monohydrate; citric acid, anhydrous; citric acid, hydrous; clove oil; cocamide diethanolamine; cocamide ether sulfate; cocamine oxide; cocoa butter; cocoa butter (pond's type 520A); cocoamphocarboxyglycinate; cocoglycerides; coconut oil; coconut oil, hydrogenated; coloring suspension; confectioners glaze; coriander oil; corn glycerides; corn oil; corn oil peg-6 esters; corn syrup; cottonseed oil; cottonseed oil, hydrogenated; cream base; creatine; creatinine; cresol, M-; croscarmellose sodium; crospovidone; cupric sulfate; cupric sulfate, anhydrous; cyclomethicone; cysteine; cysteine hydrochloride; DC antifoam AF trituration 1% on sucrose; dehydroacetic acid; dehymuls E; denatonium benzoate; deoxycholic acid; dextrates; dextrin; dextrins modified; dextrose; dextrose solution; dextrose, anhydrous; Di-Pac (97% sucrose-3% modified dextrins); diacetylated monoglycerides; diatomaceous earth; diatrizoic acid; diazolydinylurea; dibutyl phthalate; dibutyl sebacate; dichlorodifluoromethane; dichlorofluoromethane; dichlorotetrafluoroethane; dicyclohexyl-carbodiimide; diethanolamine; diethyl phthalate; diethyl sebacate; diethylamine; diglycerides; diglycol stearate; dihydroxyaluminum sodium carbonate; diisopropanolamine; diisopropyl adipate; diisopropylbenzothiazyl-2-sulfenamide; dimethicone; dimethicone 350; dimethicone 360; dimethyldioctadecylammonium bentonite; dimyristoyl lecithin; dimyristoyl phosphatidylglycerol, L-, dioctylphthalate; dipropylene glycol; disodium edisylate; disodium monooleamide sulfasuccinate; disofenin; docusate; docusate sodium; docusate sodium/sodium benzoate; Dri Klear 042; Dry Flo; Duro-Tak 280-2516; duro-tak 80-1196; dusting powder; dye beige P-1437; dye black; dye black LB-442; dye blue; dye blue #1; dye blue #2, dye brown lake; dye brown LB-292; dye brown LB-464; dye caramel; dye caramel acid proof 100; dye DC blue #2 lake; dye DC blue #6 dye DC green #3 lake; dye DC green #5; dye DC red #19; dye DC red #21 lake; dye DC red #22; dye DC red #27; dye DC red #27 aluminum lake; dye DC red #28; dye DC red #3 lake; dye DC red #30; dye DC red #30 aluminum lake; dye DC red #30 lake; dye DC red #33; dye DC red #33 lake; dye DC red #36; dye DC red #39; dye DC red #4 lake; dye DC red #40 lake; dye DC red #6; dye DC red #6 lake; dye DC red #7; dye DC red #7 calcium lake; dye DC red #7 lake; dye DC red lake; dye DC violet #2 lake; dye DC yellow; dye DC yellow #10; dye DC yellow #10 aluminum lake; dye DC yellow #10 HT lake; dye DC yellow #10 lake; dye DC yellow #5 lake; dye DC yellow #6; dye DC yellow #6 lake; dye FDC blue #1; dye FDC blue #1 aluminum lake; dye FDC blue #1 H.T. aluminum lake; dye FDC blue #1 lake; dye FDC blue #10; dye FDC blue #2; dye FDC blue #2 lake; dye FDC green #3; dye FDC green #6; dye FDC red #27 lake; dye FDC red #28; dye FDC red #3; dye FDC red #3 lake; dye FDC red #3-aluminum lake; dye FDC red #30 lake; dye FDC red #33; dye FDC red #40; dye FDC red #40 lake; dye FDC red #7 lake; dye FDC yellow #10; dye FDC yellow #10 lake; dye FDC yellow #5; dye FDC yellow #5; dye FDC yellow #5 lake; dye FDC yellow #6; dye FDC yellow #6 ht lake; dye FDC yellow #6 lake; dye gray #2982; dye green; dye green LB-482; dye green LB-603; dye green LB-883; dye green PMS-579; dye green PR-1333; dye mint green; dye ochre 3506; dye orange; dye pink; dye purple LB-562; dye red; dye red cotolene-P; dye swedish orange #2191; dye tetrarome orange; dye white; dye white coateric YPA-6-7089; dye white cotolene-P; dye white tc-1032; dye yellow; dye yellow #10; dye yellow #62; dye yellow LB 9706; dye yellow ochre; edamine; edetate calcium disodium; edetate disodium; edetate disodium, anhydrous; edetate sodium; edetic acid; egg yolk phosphatides; entsufon sodium; essence fritzbro orange; essence lemon; essence orange; ether; ethyl acetate; ethyl hexanediol; ethyl maltol; ethyl oleate; ethyl vanillin; ethylcellulose; ethylene; ethylene glycol; ethylene glycol monoethyl ether; ethylene vinyl acetate copolymer; ethylenediamine dihydrochloride; ethylparaben; ethylparaben sodium; eucalyptol; eudragit E 100; eudragit E 30 D; eudragit L 30 D; eudragit NE 30D; eudragit RL 30 D; eudragit RS 30 D; exametazine; fat, edible; fatty acid esters, saturated; fatty acid pentaerythriol ester; fatty alcohol citrate; fatty alcohols; ferric oxide; ferric oxide, red; ferrosoferric oxide; firmenich 51.226/T; flavor; flavor anise; flavor apple; flavor apricot; flavor apricot peach; flavor apricot 24829; flavor aromalok 182608; flavor aromalok 262453; flavor banana; flavor banana SAB4; flavor banana 71507; flavor banana 74546; flavor berry citrus blend 9621; flavor berry citrus blend 9756; flavor berry cream; flavor bitterness modifier 15555; flavor black cherry; flavor black currant; flavor blood orange; flavor blood orange SA; flavor blood orange 51.226T; flavor blueberry; flavor bubble gum; flavor butter vanilla; flavor buttermint toffee; flavor buttermint 24020; flavor butterscotch; flavor butterscotch F-1785; flavor candied sugar 510155U; flavor caramel fritzsche; flavor cheri-beri PFC-8573; flavor cheri-beri PFC-8580; flavor cherry; flavor cherry burgundy 11650; flavor cherry cream; flavor cherry E.P. modified 151; flavor cherry EP-3699; flavor cherry F-232; flavor cherry FMC 8513; flavor cherry IFF 13530912; flavor cherry maraschino S-3531; flavor cherry mint; flavor cherry N-2755; flavor cherry R-6556; flavor cherry raspberry; flavor cherry WL-1093; flavor cherry WL-18022; flavor cherry WL-4658; flavor cherry 11539; flavor cherry 181612; flavor cherry 3321; flavor cherry 338614; flavor cherry 349; flavor cherry 500910U; flavor cherry 594 S.D.; flavor cherry-anise; flavor cherry-anise PFC 9758; flavor chocolate; flavor chocolate cream; flavor chocolate P727; flavor citrus; flavor citrus mint; flavor citrus-vanilla; flavor cocoa; flavor coconut custard; flavor cola FMC 1574; flavor cough syrup 110257; flavor cream; flavor creme de menthe; flavor creme de menthe 14677; flavor creme de vanilla 28156; flavor curacao 50.397A; flavor custard; flavor custard 52.940/A FIR; flavor DR-119; flavor DF-1530; flavor E-472; flavor enhancer; flavor F-5397A; flavor felton 6-R-9; flavor fig; flavor fritzsche; flavor fritzsche 2102B-D or 21028-D; flavor fritzsche 75021; flavor fruit gum 912; flavor fruit mint 75588; flavor fruit punch; flavor fruit punch #28140; flavor fruit punch 14761FM; flavor fruit 01-10428; flavor fruit 84.6422; flavor fruits; flavor grape; flavor grape nectar PFC 8599; flavor grape 13403873; flavor grapefruit; flavor grenadine; flavor guarana; flavor guarana FMC-15417; flavor haverstrod ZD 49282; flavor herb alpine; flavor kola; flavor lemon; flavor lemon cream; flavor lemon lime; flavor lemon mint fritzsche 54369; flavor lemon vanilla; flavor lemon B12; flavor licorice; flavor lime; flavor mafco-magnasweet 180; flavor maque tree 377(bush); flavor MCP lemon duramone 4409A; flavor MCP lime duramone 6419; flavor mint; flavor orange; flavor orange #7679; flavor orange banana; flavor orange banana WL-18093; flavor orange natural & artificial; flavor orange terpeneless; flavor orange 13334; flavor orange-lemon terpeneless; flavor orbit serene 20340; flavor passion fruit; flavor peach; flavor peach mint fritzsche 106109; flavor peach pineapple; flavor peach pineapple FMC 14258; flavor peach 13503584; flavor peppermint; flavor peppermint stick FMC 16170; flavor peppermint 517; flavor peppermint, natural spraylene; flavor pineapple; flavor pineapple 182661; flavor pineapple-coconut; flavor raspberry; flavor raspberry A11693; flavor raspberry F-1784; flavor raspberry F-1840; flavor raspberry F-6887-S; flavor raspberry PFC-8407; flavor raspberry polak 5000064; flavor raspberry 262085; flavor raspberry 28106; flavor raspberry 954; flavor refrachessment FD-8027D; flavor rhodia pharmaceutical #RF 451; flavor root beer; flavor sherry; flavor spearmint; flavor strawberry; flavor strawberry F-5665; flavor strawberry F-5930-A; flavor strawberry F21204; flavor strawberry guarana 586.997/APO5.51; flavor strawberry microseal; flavor strawberry PFC-9626; flavor strawberry WL-16650; flavor strawberry 133.5655; flavor strawberry 14953; flavor strawberry 52312/AP; flavor strawberry 55058; flavor strawberry 5951; flavor strawberry 9843; flavor sweet; flavor sweet tone 28837; flavor tangerine; flavor tangerine fritzsche 51465; flavor tetrarome; flavor TPF 135; flavor TPF 143; flavor tropical fruit punch N&A 50432; flavor tutti frutti; flavor tutti frutti 24093FM; flavor tutti frutti 51.880/APO5.51; flavor vanilla; flavor vanilla banana; flavor vanilla creme; flavor veralock bubble gum; flavor wild cherry; flavor wild cherry NV-101-1489; flavor wild cherry PFC-147B3; flavor wildcherry 7598; flavor wintergreen; flavor wintergreen PFC 8421; flavor 57000 IU; flavor 57829/A; florasynth; flour; fluorochlorohydrocarbons; formaldehyde solution; fragrance bouquet 10328; fragrance chemoderm 6411; fragrance cream #73457; fragrance felton 066M, fragrance gardenia; fragrance givaudan ESS 9090/1C; fragrance H-6540; fragrance P O FL-147; fragrance PA 52805; fragrance pera derm D; fragrance RBD-9819; fragrance spicy metholated eugenol; fragrance ungerer N5195; fragrance unspecified; fragrance 91-122; fructose; fumaric acid; fumaric acid; galactose, D-; gamma-cyclodextrin; gelatin; gelatin 200 bloom; gellan gum; gelucire 33/01; gentisic acid; gentisic acid ethanolamide; ginger fluid extract; gluceptate sodium; gluconolactone; glucose, liquid; glucuronic acid; glutamic acid hydrochloride; glutamic acid, DL-; gluten; glycerin; glycerin hydrochloride; glycerol ester of hydrogenated rosin; glyceryl behenate; glyceryl distearate; glyceryl laurate; glyceryl oleate; glyceryl oleate/propylene glycol; glyceryl palmitate; glyceryl ricinoleate; glyceryl stearate; glyceryl stearate SE; glyceryl stearate-stearamidoethyl diethylamine; glyceryl stearate/peg-100 stearate; glyceryl stearate/peg-40 stearate; glycine; glycol stearate; glycyrrhiza; glycyrrhizin, ammoniated; guanidine hydrochloride; guar gum; gum base, chewing; gum rosin; gum, natural; herbacol; hexylene glycol; high fructose corn syrup; histidine; hydrocarbon gel, plasticized; hydrochloric acid; hydrochloric acid, diluted; hydrogen peroxide; hydroxyethyl cellulose; hydroxymethyl cellulose; hydroxypropyl cellulose; hydroxypropyl methylcellulose; hydroxypropyl methylcellulose phthalate; hydroxypropyl methylcellulose 2208; hydroxypropyl methylcellulose 2906; hydroxypropyl methylcellulose 2910; imidazolidinyl urea; imidurea; ink black; ink black A-10450; ink black A-10509; ink black A-1057; ink black imprinting FGE-1386; ink blue black A=9371; ink edible; ink edible black; ink edible gray; ink edible red; ink edible red A-8032; ink edible white; ink fine black 2202C; ink fine black 2212; ink green A-10454; ink light redwood; ink pink imprinting SB-1003; ink red A-8032; ink red S-1-9005; ink white; ink white A-8154; ink white 21-K; invert sugar; iodine; iofetamine hydrochloride; irish moss extract; iron oxide; iron oxide, brown; iron oxide, red-brown; iron oxide, yellow; isobutane; isoceteth-20; isooctylacrylate; isopropyl alcohol; isopropyl isostearate; isopropyl myristate; isopropyl palmitate; isopropyl stearate; isostearic acid; isostearyl alcohol; isotonic sodium chloride solution; jelene; kaolin; katron CG; lac resin; lactate; lactic acid; lactic acid; lactobionic acid; lactose; lactose monohydrate; lactose monohydrate, alpha, lactose, anhydrous; lactose, hydrous; lanolin; lanolin alcohols; lanolin alcohols, acetylated; lanolin cholesterols; lanolin nonionic derivatives; lanolin oil; lanolin, anhydrous; lanolin, hydrogenated; lauramine oxide; laurdimonium hydrolyzed animal collagen; laureth sulfate; laureth 23; laureth 4; lauric diethanolamide; lauric myristic diethanolamide; lauryl sulfate; lecithin; lecithin, hydrogenated soy, lecithin, soy bean, lemon oil; levomenthol; lidofenin; lime oil; limonene, DL-; linear alcohol ethylene oxide adduct; lubritab; lysine; magnesium aluminum silicate; magnesium carbonate; magnesium chloride; magnesium hydroxide; magnesium nitrate; magnesium oxide; magnesium silicate; magnesium stearate; magnesium sulfate; magnesium sulfate, anhydrous; magnesium trisilicate; maleic acid; malic acid; malic acid, DL-; maltodextrin; maltol; maltoldextrin; maltose; mannitol; mannose, D-; mebrofenin; medical antifoam emulsion C; medical antiform A-F emulsion; medronate disodium; medronic acid; meglumine; menthol; metaphosphoric acid; methacrylic acid copolymer; methanesulfonic acid; methionine; methyl acrylate-methyl methacrylate; methyl boronic acid; methyl gluceth-120 dioleate; methyl hydroexyethyl cellulose; methyl laurate; methyl salicylate; methyl stearate; methylated spirits; methylcellulose; methylcellulose 400; methylchloroisothiazolinone; methylene blue; methylisothiazolinone; methylparaben; methylparaben sodium; microcrystalline wax; mineral oil; mineral oil, light; monoglyceride citrate; monoglycerides; multisterol extract; myristic acid; myristyl alcohol; myristyl lactate; myristyl-gamma-picolinium chloride; N-(carbamoyl-methoxypolyethylene glycol 2000)-1,2-distearoyl; N-decyl-methyl sulfoxide; N-2-hydroxyethylpiperazine N′-2′-ethanesulphonic acid; N-3-chlorallyl-methenamine chloride; N,N-bis(2-hydroxyethyl)stearamide; N,N-dimethyl lauramine oxide; N,n-dimethylacetamide; neutral 01; nioxime; nipastat; nitric acid; non-pareil seed; nonoxynol; nonoxynol-15; nutmeg oil, expressed; oatmeal; octadecene-1/maleic acid copolymer; octoxynol; octoxynol-1; octoxynol-40; octoxynol-9; octyldodecanol; oil cream soda; oleic acid; oleth-10/oleth-5; oleth-2; oleyl oleate; olive oil; opacoat NA2203; opacode S-1-13001 (orange); opacode S-1-1666 (red); opacode S-1-4157; opacode S-1-4160 (blue); opacode S-1-4172 (blue); opacode S-1-4172M (blue); opacode S-1-7020; opacode S-1-7078; opacode S-1-7085 (white); opacode S-1-7534 (gray); opacode S-1-800HV (black); opacode S-1-8025 (black); opacode S-1-8081 (black); opacode S-1-8090 (black); opacode S-1-8092 (black); opacode S-1-8093 (black) opacode S-1-8095; opacode S-1-8100-HV (black); Opacode S-1-8105 (black); Opacode S-1-8106 (black); Opacode S-1088114 (black); Opacode S-1-8115 (black); Opacode S-1-9009 (brown); Opadry; Opadry (brown); Opadry (clear); Opadry (white); Opadry II Y-19-7483 (clear); Opadry II Y-22-7719 (white); Opadry OY-S-28924 (white); Opadry Y-S-17191 (brown); Opadry Y-1-1518 (pink); Opadry Y-1-2102 (yellow); Opadry Y-1-2132 (yellow); Opadry Y-1-2605 (beige); Opadry Y-1-3211 (green); Opadry Y-1-4205 (blue); Opadry Y-1-4234 (blue); Opadry Y-1-7000 (white); Opadry Y-1-7000B (white); Opadry Y-1-7006 (blue); Opadry Y-22-1452S (pink); Opadry Y-5-1244 (pink); Opadry Y-5-12584 (yellow); Opadry Y-5-14530A (pink); Opadry Y-5-1727 (red); Opadry Y-5-2028 (yellow); Opadry Y-5-2042 (yellow) Opadry Y-5-2312 (yellow); Opadry Y-5-2360 (orange); Opadry Y-5-2450 (orange); Opadry Y-5-2451 (orange); Opadry Y-5-2646 (beige); Opadry Y-5-3140 (green); Opadry Y-5-3296 (green); Opadry Y-5-4129 (blue); Opadry Y-5-4270 (blue); Opadry Y-5-4287 (blue); Opadry Y-5-7058 (white); Opadry Y-5-7068 (white); Opadry Y-5-7072 (white); Opadry Y-5-7411 (purple); Opadry Y-5-8050 (black); Opadry Y-5-9006 (brown); Opadry YS-1-11051 (green); Opadry YS-1-1107 (green); Opadry YS-1-1252 (pink); Opadry YS-1-12525-A (yellow); Opadry YS-1-12529 (yellow); Opadry YS-1-1288 (pink); Opadry YS-1-1441G; Opadry YS-14518A (pink); Opadry YS-1-1510 (pink); Opadry YS-1528 (pink); Opadry YS-1-1724 (red); Opadry YS-1-18034 (white); Opadry YS-1-1846 (red); Opadry YS-1-1847 (red); Opadry YS-1-2013 (yellow); Opadry YS-1-2065; Opadry YS-1-2074 (yellow); Opadry YS-1-2122 (yellow); Opadry YS-1-2134 (yellow); Opadry YS-1-2136 (yellow); Opadry YS-1-2167 (yellow); Opadry YS-1-2136 (yellow); Opadry YS-1-2435; Opadry YS-1-2522 (orange); Opadry YS-1-2526 (orange); Opadry YS-1-2527 (orange); Opadry YS-1-2534; Opadry YS-1-2546 (orange); Opadry YS-1-2558 (orange); Opadry YS-1-2604 (beige); Opadry YS-1-2612 (beige); Opadry YS-1-2635 (tan); Opadry YS-1-2669 (rust); Opadry YS_{—}1-3105 (green); Opadry YS-1-3130 (green); Opadry YS-1-3146 (green); Opadry YS-1-3166 (green); Opadry YS-1-4018 (blue); Opadry YS-1-4112 (blue); Opadry YS-1-4215; Opadry YS-1-4216; Opadry YS-1-4221 (blue); Opadry YS-1-4229 (blue); Opadry YS-1-4236 (blue); Opadry YS-1-4245 (blue); Opadry YS-1-4298 (blue); Opadry YS-1-4710; Opadry YS-1-6275 (orange) Opadry YS-1-6312 (yellow); Opadry YS_{—}1-6357 (yellow); Opadry YS-1-7002 (white) Opadry YS-1-7003 (white); Opadry YS-1-7006 (clear); Opadry YS-1-7027 (white); Opadry YS-1-74440 (white); Opadry YS-1-7507 (grey); Opadry YS1-7552 (grey); Opadry YS-1-7706G (white); Opadry YS-1-8325 (beige); Opadry YS-1-8345G (beige); Opadry YS-1 8619 (orange); Opadry YS-1-89193 (clear); Opadry YS-1-9012 (brown); Opadry YS 2-7013 (clear); Opadry YS-2-7063 (white); Opadry YS-3-7011 (clear); Opadry YS-3-7031 (clear); Opadry YS-3-7413 (clear); Opadry YS-5-1296 (pink); YS-5-2170 (yellow); Opadry YS-5-2370 (orange); Opadry YS-5-7042 (clear); Opaglos clear; Opaglos GS 2-0310; Opalux AS 1537 (pink); Opalux AS 1589 (pink); Opalux AS 2006 (yellow); Opalux AS 2167 (yellow); Opalux AS 2236; Opalux AS 2269 (yellow); Opalux AS 2324 (orange); Opalux AS 2336 (orange); Opalux AS 2413; Opalux AS 2498 (orange); Opalux AS 2512; Opalux AS 2676 salmon (jASper red); Opalux AS 2754; Opalux AS 3348-C (green); Opalux AS 3391 (green); Opalux AS 4208-A (blue); Opalux AS 4270 (blue); Opalux AS 5178 (green); Opalux AS 5203 (green); Opalux AS 5212 (green); Opalux AS 7000-B; Opalux AS 7000-P (white); Opalux AS 7535 (gray); Opalux AS 8050-L (black); Opalux AS 9010 (brown); opaque blue 605; opaque burgandy; opaque gray; opaque green; opaque green 1664; opaque green/flesh; opaque maroon 6 dar; opaque orange; opaque peach; opaque pink bk; opaque pink 0439; opaque red; opaque swedish orange; opaque white; opaque white 535; opaque white 536; opaque white 538; opaque yellow; opaseal; opaspray; opaspray coral; opaspray green; opaspray K-1-1230 (pink); opaspray K-1-1279; Opaspray K-1-1289 (pink); opaspray K-1-1413 (pink); opaspray K-1-1414 (pink); opaspray K-1-1455 (pink); opaspray K-1-1460; opaspray K-1-1563 (pink); opaspray K-1-1573 (lavender); opaspray K-1-1584; opaspray K-1-2013 (yellow); opaspray K-1-2088; opaspray K-1-2216-A (yellow); opaspray K-1-2228 (yellow); opaspray K-1-2240 (yellow); opaspray K-1-2275 (yellow); opaspray K-1-2301 (peach); opaspray K-1-2304 (orange); opaspray K-1-2314 (orange); opaspray K-1-2327 (orange); opaspray K-1-2330 (orange); opaspray K-1-2335 (orange); opaspray K-1-2406 (orange); opaspray K-1-2410 (orange); opaspray K-1-2430; opaspray K-1-2441 (orange); opaspray K-1-2473; opaspray K-1-2492; opaspray K-1-2533 (orange); opaspray K-1-2568 (orange) opaspray K-1-2588 (orange); opaspray K-1-2621 (brown); opaspray K-1-2626 (orange); opaspray K-1-2656 (beige); opaspray K-1-2670 (tan); Opaspray K-1-2685; opaspray K-1-3000; opaspray K-1-3147; opaspray K-1-3148 (green); opaspray K-1-3173 (green); opaspray K-1-3178 (green); opaspray K-1-3220 (green); opaspray K-1-3227; opaspray K-1-3300-A (green); opaspray K-1-3300-C (green); opaspray K-1-4136 (blue); opaspray K-1-4210-A; opaspray K-1-4227; opaspray K-1-4235 (blue); opaspray K-1-4728; opaspray K-1-4743 (lavender); opaspray K-1-4748 (purple); opaspray K-1-5024 (red); Opaspray K-1-7000 (white); opaspray K-1-70008 (white); opaspray K-1-9027 (brown); opaspray K-1-9039-L (brown); opaspray K-1-9080 (brown); opaspray K-1-9112 (brown); opaspray L-2113; opaspray L-3305 (green); opaspray L-3306 (green); opaspray L-7000 (white); opaspray M-1-7118 (white); opaspray M-1-7111-8 or M-1-7111-B; opaspray M-1-7120 (white); opaspray M-1-7301 (white); opaspray M-1-8429 (yellow); opaspray WD-1270 (pink); opaspray 3-1820; opaspray 3-1830; opatint DD-13009 (orange); opatint DD-1800 (white); orange flower oil; orange juice; orange juice, synthetic; orange oil; orange oil, terpeneless; oxidronate sodium; oxyquinoline; palm kernel oil; palm kernel oil, hydrogenated; palm oil-soybean oil, hydrogenated; palm oil, hydrogenated; palmitamine oxide; parabens; paraffin; parmacoat 606; peanut oil; pectin; peg vegetable oil; peglicol-5-oleate; pegoxol 7 stearate; pentaerythritol cocate; pentetate calcium trisodium; pentetate pentasodium; pentetic acid; peppermint; peppermint oil; perfume E-1991; perfume GD 5604; perfume tana 90/42 SCBA; perfumes; petrolatum; petrolatum, white, pharma-sweet 24052; pharmaceutical glaze; pharmacoat 606; phenol; phenol, liquefied; phenylethyl alcohol; phenylmercuric acetate; phenylmercuric nitrate; phosphate buffer; phospholipid; phosphoric acid; pine needle oil; pineapple; piperazine; piperazine hexahydrate; plastibase-50W; plusweet; polacrilin; polacrilin potassium; polistirex; poloxamer; poloxamer 188; poloxamer 331; poloxamer 407; polybutene; polydextrose; polydextrose K; polyester; polyethylene; polyethylene glycol; polyethylene glycol T-dodecyl thioether; polyethylene glycol 1000; polyethylene glycol 1450; polyethylene glycol 1500; polyethylene glycol 1540; polyethylene glycol 200; polyethylene glycol 20000; polyethylene glycol 300; polyethylene glycol 3350; polyethylene glycol 3500; polyethylene glycol 40 sorbitan diisostearate; polyethylene glycol 400; polyethylene glycol 4000; polyethylene glycol 600; polyethylene glycol 6000; polyethylene glycol 8000; polyethylene glycol 900; polyethylene oxide; polyethylene terephthalates; polyglactin; polyglyceryl-10 tetralinoleate; polyisobutylene; polyisobutylene 1,200,000; polylactide; polymers; polyols; polyoxyethylene-polyoxypropylene 1800; polyoxyethylene alcohols; polyoxyethylene fatty acid esters; polyoxyethylene propylene; polyoxyethylene sorbitan monoisostearate; polyoxyl castor oil; polyoxyl distearate; polyoxyl glyceryl stearate; polyoxyl lanolin; polyoxyl stearate; polyoxyl 100 glyceryl stearate; polyoxyl 100 stearate; polyoxyl 15 cocamine; polyoxyl 150 distearate; polyoxyl 2 stearate; polyoxyl 20 stearate; polyoxyl 35 castor oil; polyoxyl 40 castor oil; polyoxyl 40 hydrogenated castor oil; polyoxyl 40 stearate; polyoxyl 50 stearate; polyoxyl 60 castor oil; polyoxyl 75 lanolin; polyoxyl B stearate; polyoxypropylene 15 stearyl ether; polyoxypropylene 26 oleate; polypropylene; polypropylene glycol; polysaccharide; polysiloxane; polysorbate; polysorbate 20; polysorbate 40; polysorbate 60; polysorbate 80; polysorbate 80; polysorbate 85; polyvinyl acetate; polyvinyl acetate phthalate; polyvinyl alcohol; polyvinylacetal; polyvinylpyridine; polyvinylpyrrolidone ethylcellulose; poppy seed oil; potassium acetate; potassium carbonate; potassium chloride; potassium citrate; potassium hydroxide; potassium metabisulfite; potassium phosphate, dibasic; potassium phosphate, monobasic; potassium polacrilin; potassium sorbate; povidone; povidone K25; povidone K29-32; povidone K30; povidone K90; promalgen type G; promulgen D; promulgen G; propane; propenyl guaethol; propyl gallate; propylene carbonate; propylene glycol; propylene glycol alginate; propylene glycol diacetate; propylene glycol monolaurate; propylene glycol monostearate; propylparaben; propylparaben sodium; prosweet; prosweet 604; protamine sulfate; protein hydrolysate; RA-2397; RA-3011; rosin; saccharin; saccharin calcium; saccharin sodium; saccharin sodium anhydrous; satialgine H; sea spen; sesame oil; shellac; shellac P.V.P. solution no. 4; silastic brand medical grade tubing; silastic medical adhesive, silicone type a; silica gel; silica, diatomaceous; silicon; silicon dioxide; silicone; silicone emulsion; silicone/polyester film strip; simethicone; simethicone emulsion; simethicone MDX4-4036; soap; soap, eiderdown; sodium acetate; sodium acetate, anhydrous; sodium acid pyrophosphate; sodium alginate; sodium alkyl sulfate; sodium aminobenzoate; sodium ascorbate; sodium benzoate; sodium bicarbonate; sodium bilsulfate; sodium bisulfite; sodium borate; sodium borate decahydrate; sodium carbonate; sodium carbonate; sodium carbonate hydrate; sodium carragenate; sodium cellulose; sodium chlorate; sodium chloride; sodium chloride injection; sodium chloride injection, bacteriostatic; sodium citrate; sodium citrate anhydrous; sodium citrate dihydrate; sodium desoxycholate; sodium dithionite; sodium dodecylbenzenesulfonate; sodiumformaldehyde sulfoxylate; sodium hexametaphosphate; sodium hydroxide; sodium hypochlorite; sodium iodide; sodium L-cysteinate hydrochloride; sodium L-lactate; sodium lactate; sodium laureth sulfate; sodium laureth-5 sulfate; sodium lauroyl sarcosinate; sodium lauryl sulfate; sodium lauryl sulfoacetate; sodium metabisulfite; sodium phosphate; sodium phosphate dihydrate; sodium phosphate, dibasic; sodium phosphate, dibasic, anhydrous; sodium phosphate, dibasic, dihydrate; sodium phosphate, dried; sodium phosphate, monobasic; sodium phosphate, monobasic, monohydrate; sodium phosphate, tribasic; sodium propionate; sodium pyrophosphate; sodium pyrrolidone carboxylate; sodium starch glycolate; sodium stearyl fumarate; sodium succinate; sodium sulfate; sodium sulfate, anhydrous; sodium sulfite; sodium tartrate; sodium thioglycolate; sodium thiosulfate; sodium thiosulfate, anhydrous; sodium trimetaphosphate; solulan; sorbic acid; sorbitan monolaurate; sorbitan monooleate; sorbitan monopalmitate; sorbitan monostearate; sorbitan sesquioleate; sorbitan solution; sorbitan trioleate; sorbitol; sorbitol solution; soybean oil; soybean oil, hydrogenated; spearmint oil; spectrablend CSL-15764 (blue); spermaceti; squalane; stannous chloride; stannous chloride, anhydrous; stannous fluoride; stannous tartrate; starch; starch 1500, pregelatinized; starch 1551; starch, corn; starch, potato; starch, pregelatinized; starch, pregelatinized corn; starch, pregelatinized tapioca; starch, rice; starch, tapioca; starch, wheat; stear-o-wet C; stear-o-wet M; stearalkonium chloride; stearalkonium hectorite/propylene carbonate; stearamidoethyl diethylamine; steareth; steareth-10; steareth-100; steareth-2; steareth-21; stearic acid; stearyl alcohol; stearyl citrate; succimer; succinic acid; sucrose; sucrose polyesters; sucrose stearate; sucrose syrup; sugar compressible; sugar confections; sugar fruit fine; sugar liquid type #0; sugar non-pareil seeds; sugar/starch insert granules; sugars (unidentified); sulfuric acid; sulfurous acid; suppocire; synchron oral carrier; synchron oral carrier vehicle type EM; tagatose; talc; tall oil; tallow glycerides; tartaric acid; tartaric acid, DL-; tenox; terpene resin; terpineol, alpha; tetrakis(1-isocyano-2-methoxy-2-methyl-propane)-copper(I) TE; thiazoximic acid; thimerosal; thioglycerol; thymol; timing solution clear N-7; titanium dioxide; tocopherol; tragacanth; triacetin; tribehenin; trichloromonofluoromethane; trideceth 10; triethyl citrate; triglyceride, synthetic; trihydroxystearin; trilaneth-4 phosphate trilaureth 4 phosphate; trimyristin; tristearin; trithiazoximic acid; triton X-200 sodium salt of alkylauryl polyether sulfonate; trolamine; trolamine lauryl sulfate; tromethamine; tyloxapol; Union 76 amsco-res 6038; unspecified ingredient; urea, vanillin; vegetable oil; vegetable oils, hydrogenated; vegetable shortening; vinyl acetate-crotonic acid copolymer; vinyl chloride; viscarin; vitamin E; water for injection, bacteriostatic; wax; wax blend; wax, dehydag; wax, emulsifying; wax, white; wax, yellow; witepsol E-85; witepsol W-35; xanthan gum; zarzarol; zein; zeolex; zinc acetate; algae meal dried; alumina; aluminum powder; annatto; annatto extract; beet juice; beet dehydrated; beet powder; benzamide N N-(9,10-dihydro-9,10-dioxo-1,5-anthracenediyl)BIS-; benzenetriol, 2-[(2,5-diethoxy-4[(4-methylphenyl)thiophenyl)]; beta-apo-8′-caroteneal; beta carotene, natural & synthetic; bismuth citrate; bismuth oxychloride; bixin; bronze powder; calcium carbonate; canthaxanthin; carbazole violet; carmine-carotene; carrot oil; chlorophyllin-copper complex; chlorophyllin-copper complex, oil soluble; chromium hydroxide, green; chromium oxide greens; chromium-cobalt-aluminum oxide; CI VAT orange 1; citrus red #2; cochineal extract; copper metallic powder; corn endosperm oil; cottonseed flour, toasted, partially defatted & cooked; dihydroxyacetone; dinaphtho(2,3-A 2′3-C]carbazole-5,10,15,17,22,24-hexone, 16,23-di-hydro; disodium edta-copper; dye caramel; dye DC blue #4; dye DC blue #9; dye DC brown #1; dye DC green #6; dye DC green #8; dye DC orange #10; dye DC orange #11; dye DC orange #4; dye DC orange #5; dye DC red #17; dye DC red #31; dye DC red #34; dye DC yellow #11; dye DC yellow #7; dye DC yellow #8; dye ext DC violet #2; dye ext DC yellow #7; dye ext DC lakes; dye FDC yellow #7; ferric ammonium citrate; ferric ammonium ferrocyanide (iron blue); ferric ferrocyanide (iron blue); ferrous gluconate; fruit juice; grape color extract; grape skin extract (enocianna); guanin (pearl essence); guayazulene (azulene); henna; iron oxides; iron oxide, synthetic; lead acetate; manganese violet-methyl umbilliferone; mica; norbixin; orange B; paprika & paprika oleoresin; phthalocyaninato-2-copper; phthalocyanine green; poly(hyoroxyethylmethacrylate); dye copolymers; pyrogallol; pyrophyllite; pyrophyllite aluminum silicate; reactive blue #19; riboflavin; safferon (crocus sativus L); silver; tagetes meal extract (aztec marigold); talc; titanium dioxide; tumeric & tumeric oleoresin; ultramarine green; ultramarine pink; ultramarine red; ultramarine violet; vegetable juice; xanthophyll; zinc oxide; 4-(2,4-dimethylphenyl) azol-2-4-dihydro-5-methyl-2 phenyl-3H-pyrazol-3-one; 5,9,14,18-anthrazine; 9,10-anthracenedione, 1,4-bis[2-methylphenyl)amino]; 6-ethoxy-2-(6-ethoxy-3-oxo-benzo[b]thein-2-(3H)-ylidene)benzo[b]thiophen-3-(2H)-one; 1,4-bis[4-(2-methacryloxyethyl)phenlamino]anthraquinone; 16,23-dihydrodinaptho[2,3→2′,3′-I]napth[2′3′6.7]indolo[2,3-C]carbazole-5,10,15,17,22,24-hexone; N.N-(9,10-dihydro-9,10-dioxo-1,5-anthracenediyl)bisbenzamide; 7,16-dichloro-6,15-dihydro-5,9,14,19-anthrazinetetrone; 15,17- or 16,17-dimethoxdinaptho[1,2,3-CD, 3′,2′,1′IM]perylene-5,10,dione; 2-{[2,5-diethoxy-4[(4methylphenyl)thiol]phenyl]azo]-1,3,5-benzenetriol; 1,4-BIS[(2-methylphenyl)amino]-9,10-anthracenedione.

Additionally, distribution agents and blood modifiers can be selected from the following functional categories, and titrated for optimal concentrations based on the information and techniques described elsewhere herein: acidifying agents (acidulants); additives color (coloring agents); adsorbents; aerosol propellants; air displacement agents; alkalizing agents; anticaking agents; anticoagulants; antimicrobial preservatives/antiseptics/disenfectants; antioxidants; bases; binders; buffering agents; lubricants (for capsule/tablet); chelating agents; coating agents; controlled release vehicles; dessicants; detergents; diluents (for capsule/tablet); disintegrants (for capsule/tablet); dispersing agents; dissolution enhancing agents; drug deliver systems; dusting powders; dyes (coloring agents); emolients; emulsifying agents; emulsion stabilizers; extened release agents; fillers; film forming agents; flavor enhancers (flavoring agents); flow enhancers; gelling agents; glidants; granulating agents; humectants; lubricants; medical dusting powders; mucoadhesives; oleaginous vehicles; plasticizers; polishing agents; preservatives; sequestering agents; solubilizing agents; solvents; stabilizing agents; stifferning agents; surfactants (ionic, nonionic); suspending agents; sweetening agents; thickening agents; tonicity agents; viscosity increasing agents; water miscible cosolvents; water softeners. In that regard, Oleaginous vehicles inlcudes, but are not limited to, canola oil; corn oil; cottonseed oil; ethyl oleate; mineral ail; peanut oil; sesame oil; soybean oil. Lubricants include, but are not limited to, calcium stearate; canola oil; glyceryl palmitostearate; hydrogenated vegetable oil, type I; magnesium oxide; mineral oil; poloxamer; polyethylene glycol; polyvinyl alcohol; sodium benzoate; sodium lauryl sulfate; sodium stearyl fumarate; stearic acid; talc; zinc stearate. Surfactants (non ionic and ionic) include, but are not limited to anionic (ocusate sodium; sodium lauryl sulfate), cationic: (certrimide), and nonionic {polyoxyethylene fatty acid esters (polysorbates) (polysorbate 20, 40, 60 used orally); sorbitan ester (sorbitan fatty acid esters)}. Wetting agents include, but are not limited to, benzalkonium chloride; castor oil, polyethoxylated; docusate sodium; ether, polyoxethylene; poloxamer; polyoxethylene esters; polyoxyethylene fatty acid ether (polysolbrate); polyoxyethylenes, stearates; sodium lauryl sulfates; sorbitan esters (sorbitan fatty acid esters). Solubilizing agents include, but are not limited to, benzalkonium chloride; castor oil, polyethoxylated; cyclodextrins; ethers, polyoxyethylene; glyceryl monostearate; lecithin; poloxamer; polysorbates; polyoxyethylene stearates; sorbitan esters (sorbtitan fatty acid esters); stearic acid,. Water miscible cosolvents include, but are not limited to, propylene glycol.

Information pertinent to the choice of distribution agents, for example as regards the choice of nontoxic agents, is available in the following references, all of which are incorporated herein by reference:

Food Chemicals Codex, Fourth Edition, (National Academy Press: Washington, D.C., 1996);

Food Chemicals Codex, First Supplement to the Fourth Edition, (National Academy Press: Washington, D.C., 1997);

Food Chemicals Codex, Second Supplement to the Fourth Edition, (National Academy Press: Washington, D.C., 2000);

Huang, Kee Chang, The Pharmacology of Chinese Herbs, Second Edition, (CRC Press: Boca Raton, 1999);

Kibbe, Arthur H., Handbook of Pharmaceutical Excipients, Third Edition, (American Pharmaceutical Association: Washington, D.C., 2000);

Japanese Pharmaceutical Excipients, Main Edition plus Supplements 1-3, English Language Edition, 1996;

Japanese Pharmaceutical Excipients Directory, English Language Edition, 1996.

Pharmacopoeia of the People's Republic of China, English Language Edition, Vols. 1-2, (Pharmacopoeia Commission of the Ministry of Public Health, 1997); and

The Inactive Ingredient Guide of the Division of Drug Information Resources, Food and Drug Administration, Rockville, Md. 20857.

Suitable smoking deterrent agents include, but are not limited to, nicotine, buprorion, fasudil, ziconotide, RSR13, and any derivatives and/or combinations thereof.

Suitable nutritional supplements include, but are not limited to, amino acid preparations, minerals, electrolytes, vitamins, calcitriol, and any derivatives and/or combinations thereof.

Suitable anti-infective agents include, but are not limited to, terbinafine, ticarcillin disodium, cefixime, meropenem, cefprozil, levofloxacin, cefpodoxime proxetil, imipenem, cefuroxime axetil, trovafloxacin, mupirocin, stavudine, didanosine, nevirapine, lamivudine, zidovudine, valcyclovir, ganciclovir, nefiracetam, and any derivatives and/or combinations thereof.

Suitable central nervous system agents include, but are not limited to, remifentanil, sevoflurane, tiagabine, topiramate, lamotrigine, naratriptan, bromocriptine, tolcapone, oxaprozin, diclofenac and misoprostol, nabumetone, granisetron, fasudil, dotarizine, ziconotide, RSR13, zonisamide, BMS204352, foropatant, oxcarbazepine, tropisetron and any derivatives and/or combinations thereof.

Suitable anti-neoplastic agents include, but are not limited to, irinotecan, topetecan, anastrozole, nilutamide, cladribine, gemcitabine, letrozole, vinorelbine, epirubicin, and any derivatives and/or combinations thereof.

Suitable endocrine agents include, but are not limited to, raloxifene, calcitonin, somatotropin, recombinant somatropin, tolterodine, temiverine, meluadrine tartrate, and any derivatives and/or combinations thereof.

Suitable gastrointestinal agents include, but are not limited to, lansoprazole, misoprostol, ropivacaine, and any derivatives and/or combinations thereof.

Suitable anti-asthmatic and pulmonary agents include, but are not limited to, bambuterol, israpafant, foropatant, rupatadine, levosalbutamol, ARC68397AA, salbutamol (powder) (Chiesi & Astra Zeneca), salbutamol (inhalation) (Astra Zeneca & Aventis), salbutamol (oral), salbutamol (powder inhilation) (Astra Medici & IVAX), formoterol, salmeterol/fluticasone propionate, salmeterol MDI dose counter, salmeterol (inhilation) (GSK), salmeterol hydrofluoroalkane, budesonide/formoterol, olopatadine, and any derivatives and/or combinations thereof.

Suitable opthalmic agents include, but are not limited to, levobetaxolol, levobunolol, latanoprost/timolol, ketotifen, and any derivatives and/or combinations thereof.

Suitable chelating agents include, but are not limited to, desferoxamine, and any derivatives, and/or combinations thereof.

Suitable granulocyte colony stimulating factors include, but are not limited to, leukine, sargramostin, GM-CSF, and any derivatives and/or combinations thereof.

Reduced viscosity can reduce the permeability of leaky junctions, thus reducing intimal injury. It should be noted that enhanced permeability of E-cells causes influx of lipids and macromolecules. Reduced viscosity does reduce the magnitude of high shear stress at the flow divider 255 of an arterial bifurcation because wall shear stress is proportional to blood viscosity.

There is also a relationship between blood viscosity and thrombosis. Thrombosis often occurs in the later stages of the arterial disease. Blood viscosity may be indirectly related to thrombogenesis. By reducing blood viscosity, the occurrence of thrombosis can be reduced because reduced blood viscosity increases flow velocity. Coagulability or thrombogenicity of blood indicates the blood's tendency to coagulate, form thrombi, aggregate platelets or clot. Thus, as shown in FIG. 11, by measuring both the absolute and effective blood viscosity profiles and monitoring the angle between the two profiles, θ, one can quantitatively evaluate the blood's tendency to form thrombi, as discussed in application Ser. No. 09/501,856.

As mentioned earlier and as shown in FIGS. 1 and 2, four additional analyzers have been introduced, namely, hematocrit analyzer 300, plasma viscosity analyzer 400, surface tension analyzer 500 and red blood cell deformability analyzer 600. Each of these analyzers operate independently of the blood viscometers 20/120 but take advantage of sharing the single intubation of the living being via circulating blood conveying means 26.

With regard to the hematocrit analyzer 300 (FIGS. 6-8), there is a need to monitor the level (percentage) of hematocrit of blood on a real time base. As blood is drawn out of a living being, if one can measure or monitor the hematocrit, it can improve health care quality, diagnostic capability and treatment.

Currently, hematocrit (which is defined as the volume percentage of red blood cells in whole blood wherein the hematocrit of a normal healthy individual is approximately 40%-45%) is measured using a small capillary tube where a small amount of blood is filled from one end, and the end is closed by an amorphous, dough-like material. Using a (micro) centrifuge, cells from blood are separated from plasma and the volume of the cells is read in terms of percentage, called hematocrit.

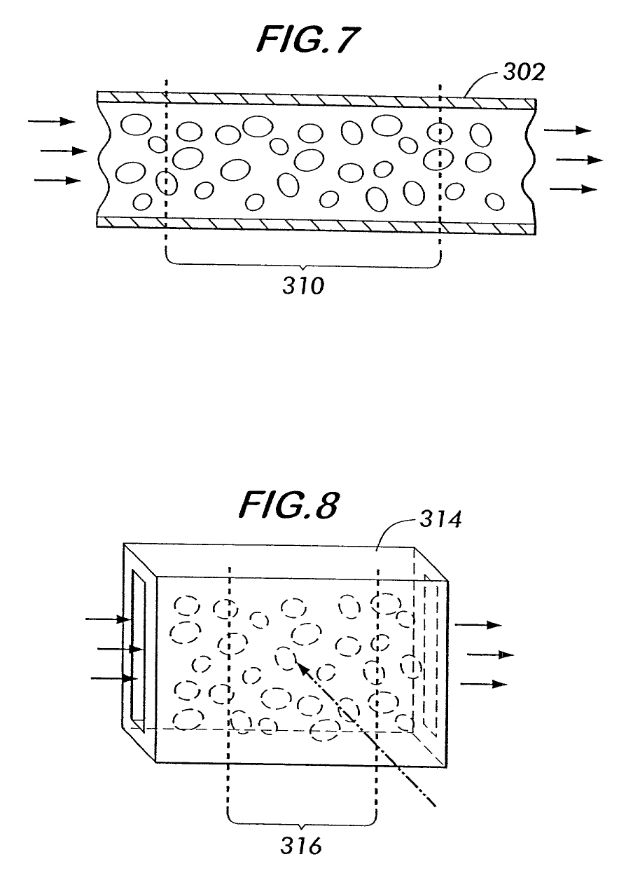

In contrast, the present invention implements a hematocrit analyzer 300 which utilizes an optical non-contact method. Blood is diverted from the circulating blood conveying means 26, through the valve 700 and into the hematocrit analyzer 300. In particular, the blood sample flows through a transparent capillary tube 302 of approximately 20-50 μm ID. A pulsing light 304 (e.g., a strobe light) provides illumination and optically “freezes” the motion of cells inside the tube 302. A red blood cell detector 305 is used to count the red blood cells and may comprise a CCD camera microscope 306 and an image processing means 308. Multiple imaging frames, e.g., 10 frames/second, can be captured by the CCD (charge coupled device) camera/microscope 306 (e.g., a CCD having 300 dpi-83μ pixel resolution available from ScanVision Inc. of San Jose, Calif.) and processed through the image processor 308 which includes image processing software (e.g., conventional CCD acquisition software available with the ScanVision Inc. CCD mentioned previously). The image processor 308 identifies cells and counts the number of cells in a given window 310 (FIG. 7). The given window has a predetermined volume. Since one can calculate the total volume and cells from cell count, one can estimate the volume percentage of cells, thus hematocrit. The total volume and cell count can then be transmitted to a computer 312, or to the microprocessor 58 (FIGS. 1 and 2) in the viscometer 20/120.

The new method utilized by the hematocrit analyzer 300 can easily be validated by comparing the hematocrit data generated from the analyzer 300 with those obtained from the conventional method such as the microcentrifuge method described earlier.

FIG. 8 depicts an alternative lumen to the capillary tube 302. In particular, a rectangular glass tube or lumen 314, which is readily available off-the-shelf, can be used and a predetermined window 316 can be established for conducting the total volume and cell count.

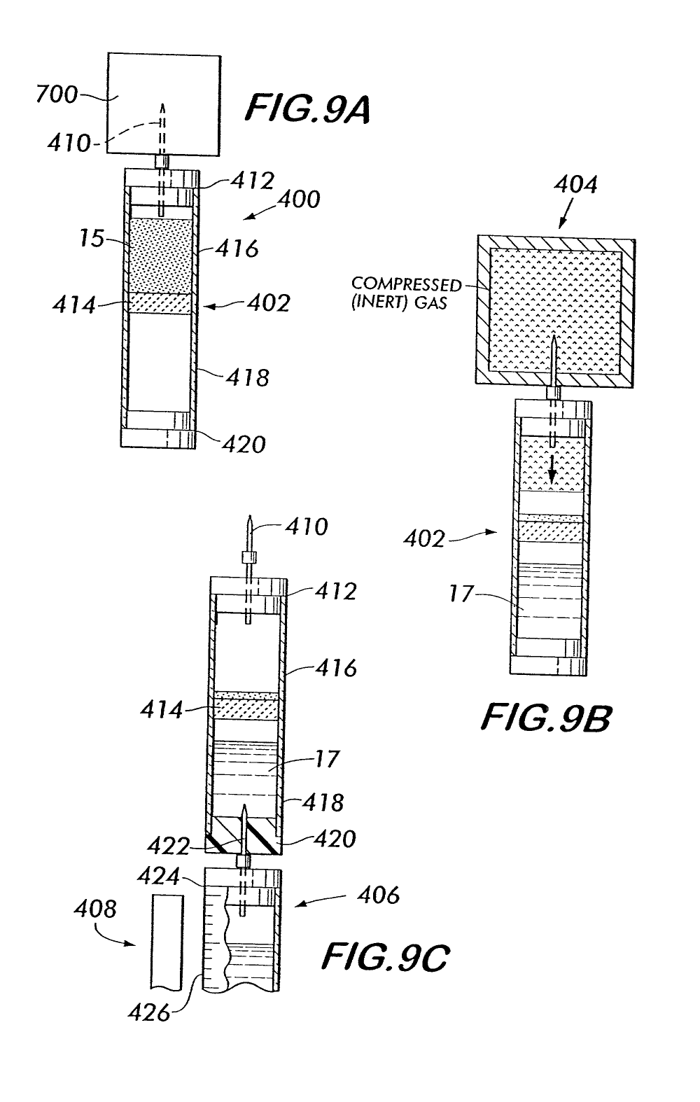

FIGS. 9A-9C depict portions of the plasma viscosity analyzer 400 which basically comprises a first vacutainer 402, an optional high pressure source 404, a second vacutainer 406 and an automated volume reader 408. Unlike the conventional way plasma is obtained, e.g., utilizing a centrifuged blood sample, in the plasma viscosity analyzer 400, a portion of the circulating blood is diverted therein from the living being using the single intubation of the living being via the circulating blood conveying means 26. In particular, as can be seen from FIG. 9A circulating blood is diverted to the plasma viscosity analyzer 400 via the valve 700. A lumen 410 (e.g., a 21 gauge needle) releasably fits into the valve 700. The other end of the lumen 410 passes through a rubber membrane or plug 412 in the top portion of a first vacutainer 402. Disposed inside at the center of the vacutainer 402 is a porous medium, e.g., a membrane filter 414, which separates the vacutainer 402 into an upper chamber 416 for collecting the circulating blood 15 therein and a lower chamber 418 that initially comprises a vacuum.

The membrane filter 414 separates cells only, but not fibrinogen. A filter membrane used for ultra-filtration with a pore size of approximately 0.1 μm should be suitable for this purpose.

Once the circulating blood conveying means 26 is in fluid communication with the plasma viscosity analyzer 400 via valve 700, blood 15 flows into the upper chamber 416. Under the influence of gravity and the pressure differential, the red blood cells are separated from the plasma 17 (FIG. 9B) via the membrane filter 414, i.e., the red blood cells remain in the upper chamber 416, with the plasma 17 being collected in the lower chamber 418.

Furthermore, if the vacuum in the lower chamber 418 is not sufficient to pull plasma through the membrane 414, to accelerate this separation process, the first vacutainer 402 can be disengaged from the valve 700 and coupled to a high pressure source 404 (FIG. 9B, e.g., compressed air). The high pressure source 404 forces the collected blood 15 against the porous membrane 414 and thereby separates the plasma 17 much more quickly. It is important to note that plasma 17 is a Newtonian fluid, therefore the viscosity thereof does not vary with shear rate. Thus, to determine the plasma viscosity, it is only necessary to obtain one shear rate, i.e., it is not necessary to monitor the change in height of a column of plasma.

As shown in FIG. 9C, once the red blood cells and plasma are separated, the first vacutainer 402 is disengaged from the valve 700 (or from the high pressure source 404, if used). A second vacutainer 404, having graduations indicating different volume levels, is coupled to the first vacutainer 402. In particular, a lower rubber membrane or plug 420 of the first vacutainer 402 is pierced by another lumen 422 (e.g., a 21 gauge needle). The other end of the lumen 422 is disposed through a rubber membrane plug 424 of the second vacutainer 404. With the first lumen 410 is exposed to atmosphere (i.e., zero gauge pressure) as shown in FIG. 9C, the pressure above the plasma 17 is atmospheric pressure; furthermore, the second vacutainer 404 comprises a predetermined vacuum level. Because of this pressure differential (Δp) between the two vacutainers 402/404, when the second lumen 422 punctures the membrane/plug 420, the plasma 17 is forced down out of the first vacutainer 402 down into the second vacutainer 404 via the lumen 422.

As mentioned earlier, since plasma is a Newtonian fluid, plasma viscosity can be determined from a single shear rate, according to the equation:

$$Q = \frac{π {d}^{4} Δ P}{128 {μ}_{P} L}$$

where

L=second lumen 422 length;

d=second lumen 422 inside diameter;

ΔP=pressure difference between the two vacutainers 402/404 as shown in FIG. 9C (i.e, pressure levels are predetermined and vary depending on the accumulated plasma amount, thus mathematically estimated, no need to measure)

Q=flow rate, or volume/time; and

μ_{P}=plasma viscosity.

Thus, μ_{P}, is given by

$${μ}_{P} = \frac{π {d}^{4} Δ P}{128 QL} .$$

By measuring the volume of plasma 17 accumulated over a given period (e.g., 1 minute) in the second vacutainer 406 using the predetermined/premarked lines 426 on the side wall of the vacutainer 406 using a manual method or an automated volume reader 408, the plasma viscosity can be calculated. Alternatively, one can measure the mass of the second vacutainer 406 over a given period (e.g., 1 minute) from which one can estimate the plasma viscosity.

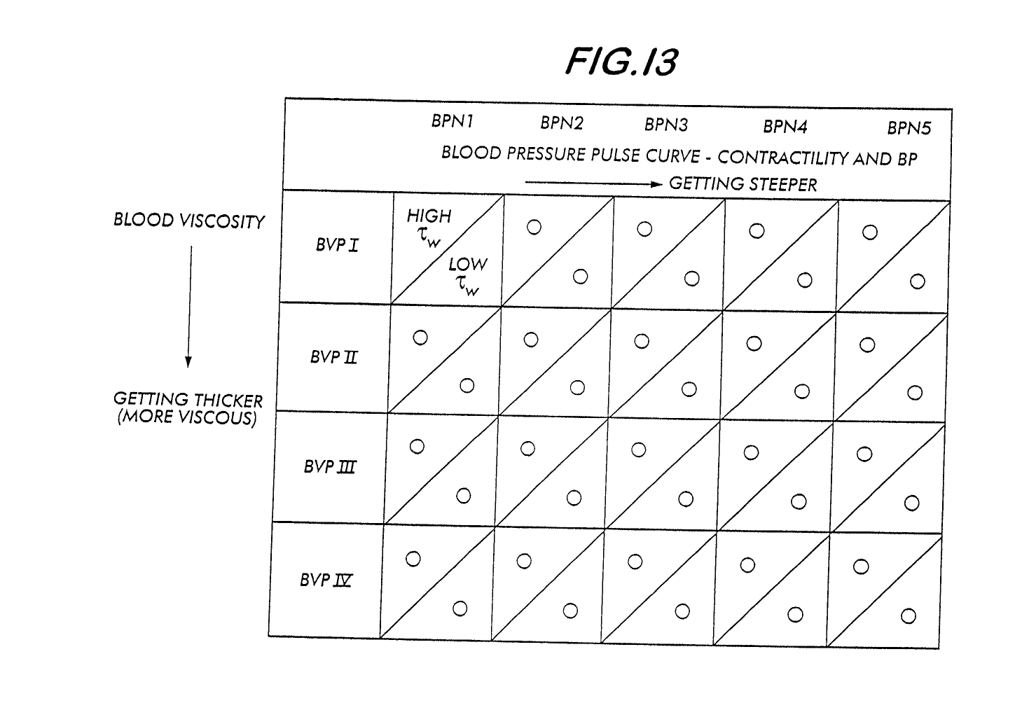

In another aspect of this invention, it is desirable to utilize blood pressure, the heart's pressure pulse curve, and blood viscosity information in order to estimate wall shear stress in high and low shear areas of a (coronary) bifurcation. As discussed earlier with regard to FIG. 4, the circulating blood flows at the (1) wall 256 opposite the flow divider 255 and at the (2) proximal side 265 of the branch vessel 255 are experiencing low wall shear stress whereas the distal side 269 of the branch vessel 255 is experiencing high shear wall stress. In particular, the inventors have developed a table (FIG. 13) showing both high and low wall shear stress that is based on computational fluid dynamic (CFD) modeling of the coronary bifurcation flow. Two parameters are used, namely BPN (i.e., blood pressure number) and BVP (blood viscosity parameter), which will be defined later below.

To use the wall-shear-stress table, it should be understood that it is practically impossible to calculate oscillating wall shear stress based on BPN and BVP data on a real time basis with current high speed computers. Furthermore, it may not be necessary to have complicated pulsatile flow information for ordinary clinical diagnostics and treatments of various diseases such as hypertension, diabetes, etc.

The table (FIG. 13) provides the high and low shear data as soon as the BPN and BVP data become available. For example, if a patient has a BVP III level and BPN5 level, one can read from the table the corresponding values of high wall shear stress (high τ_{W}) and low wall shear stress (low τ_{W}). The objective of any drug administration and clinical treatment is to move a patient condition from the lower right corner (i.e., the worst wall shear stress conditions) to the upper left corner (i.e., the ideal wall shear stress conditions).

The definitions of BVP and BPN are discussed next. To understand the definition of BVP, it is necessary to discuss the absolute viscosity profile and the effective viscosity profile in view of FIGS. 10-12B. As disclosed in application Ser. No. 09/501,856, once the height vs. time data is collected from changing column levels in the riser tubes R1 and R2, that data can be segmented into a first shear rate range A (e.g., 320s⁻¹ to 1s⁻¹) and a second shear rate range B (e.g., 1s⁻¹ to 0.02s⁻¹). It should be understood that the particular shear rate selected to define the end of range A and the beginning of range B, e.g., 1s⁻¹, is by way of example only and not limitation; thus, it is within the broadest scope of this invention to cover any number of shear rates that define the end of range A and the beginning of range B.

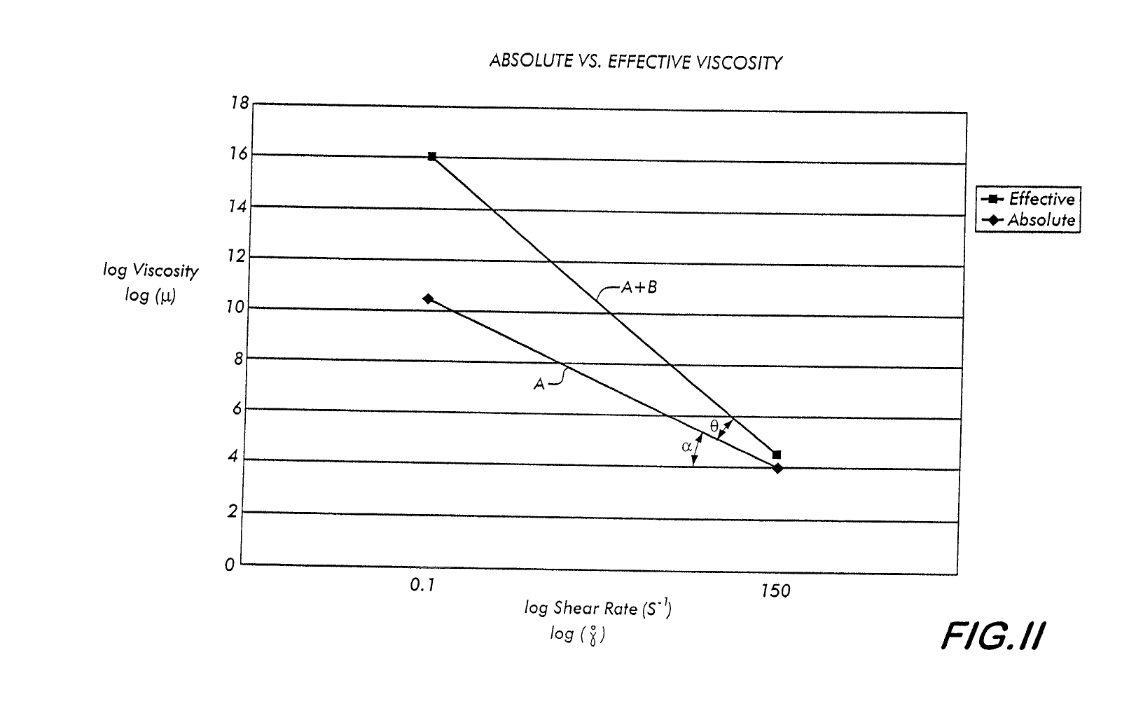

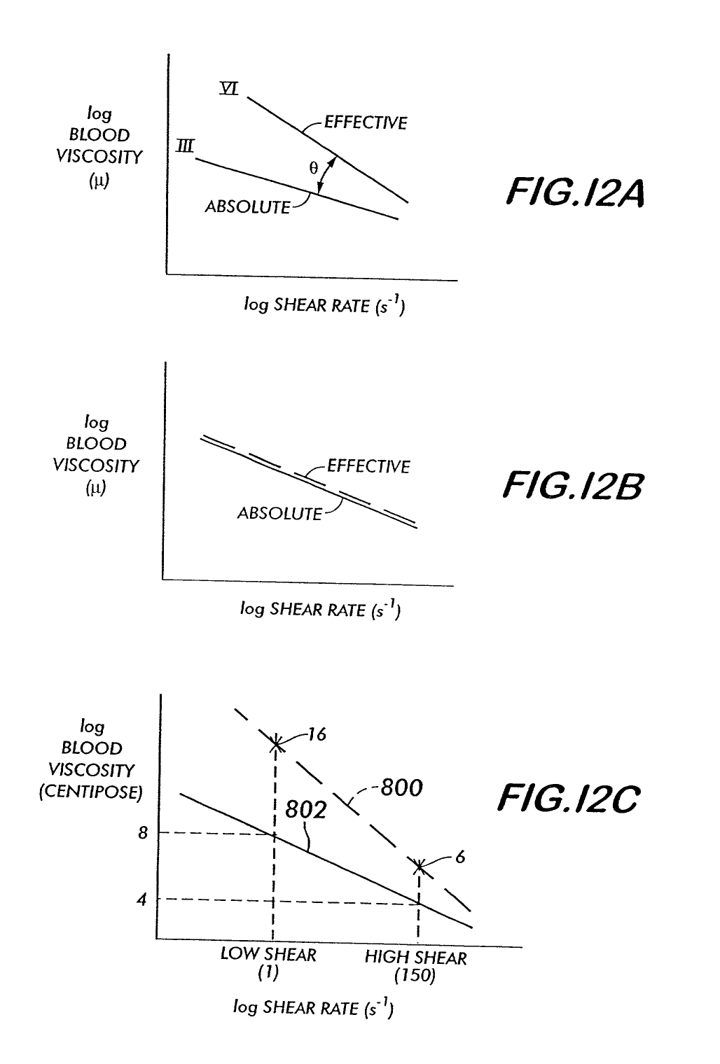

When the blood viscosity is plotted over time in a log-log scale, the viscosity profile over the first shear rate range (A) is called the “absolute viscosity profile” and the viscosity profile over the first and second shear rate ranges (A+B) is called the “effective viscosity profile” (see FIG. 11). As also disclosed in application Ser. No. 09/501,856, the angle θ (FIG. 12A) formed between the absolute viscosity profile and the effective viscosity profile can be used as an indicator of blood parameters. As is also discussed in application Ser. No. 09/501,856, it is desirable to minimize the angle θ as shown in FIG. 12B by providing medications, changing the living being's lifestyle, or both, etc.

The blood viscosity parameter (BVP) is a value that is determined from comparing the effective viscosity profile 800 of the living being under test (UT) to the effective viscosity profile 802 of a normal healthy living being, e.g., a healthy young boy, as shown in FIG. 12C. For a normal healthy person, BVP varies between approximately 5-10 and is defined as:

$$BVP = ( \frac{{μ}_{effective}}{{μ}_{absolute}} - 1 ) \cdot 50 + ( \frac{{μ}_{150}}{4} ) \cdot 2 + ( \frac{{μ}_{1}}{8} ) \cdot 3$$

where:

μ_{effective} is the the effective viscosity profile 800 of a living being UT;

μ_{absolute} is the absolute viscosity profile and the effective viscosity profile 802 of a normal healthy person, in a log-log graph;

μ₁₅₀ is the living being UT's circulating blood viscosity measured at \dot{γ}=150 s⁻¹; and

μ₁ is the living being UT's circulating blood viscosity measured at \dot{γ}=1 s⁻¹.

The first term

$$( \frac{{μ}_{effective}}{{μ}_{absolute}} - 1 ) \cdot 50$$

is known as the “effective/absolute viscosity” term and represents blood's thrombotic tendency. The second term

$$( \frac{{μ}_{150}}{4} ) \cdot 2$$

is known as the “high shear effect” term and the third term

$$( \frac{{μ}_{1}}{8} ) \cdot 3$$

is known as the “low shear effect” term.

The denominators of the high shear effect term and the low shear effect term are used as references and are the viscosity values (4 and 8 centipoise) from a healthy young boy at the shear rates of 150 s⁻¹ and 1 s⁻¹, respectively (see FIG. 12C). For diabetes, the high shear effect term can be much greater. A weighting factor of “3” is used with the low shear effect term because the low shear viscosity is often a direct cause of arterial disease. Furthermore, since μ₁ for the subject is often much greater than 8 centipoise for most adults, the low shear effect term can be the largest contributor among the three terms. A weighting factor of “2” is used with the high shear effect term since the effect of high shear on atherosclerosis is less than that of low shear viscosity.

With regard to the BPN, the BPN can be defined as an average blood pressure term (i.e., the average value of the systole and diastole) and a contractility of the heart (COH) term.

Once a BVP and a BPN is determined for any particular living being, these values can be immediately referenced according to the table shown in FIG. 13 and the high and low wall shear stress indicated. Depending on the patient's particular BVP/BPN, the physician and/or specialist can then devise a regimen of drugs and/or lifestyle changes (as mentioned previously) to move the patient's cardiovascular system toward the upper left corner of the table in FIG. 13.

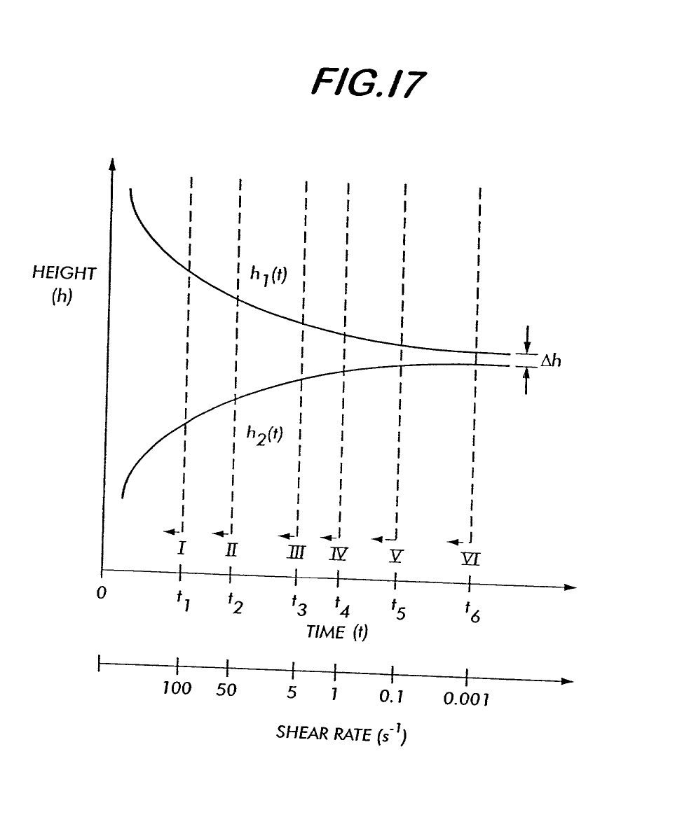

As mentioned earlier, the h₁(t) and h₂(t) data/curves of the viscometers 20/120 can be segmented into two shear rate regions (A and B) and from which an absolute viscosity profile and an effective viscosity profile can be obtained. These h₁(t) and h₂(t) data/curves can be further segmented into a plurality of regions (see FIG. 17), resulting in a plurality of viscosity profiles (see FIG. 18A), and two of which are the absolute viscosity profile (III, in FIGS. 12A and 18A) and the effective viscosity profile (VI, in FIGS. 12A and 18A). As an example, as shown in FIG. 17, the h₁(t) and h₂(t) data/curves have been segmented into six regions.

The determination of the blood viscosity profile for each segment is in accordance with the equations set forth in application Ser. No. 09/501,856 and application Ser. No. 09/439,795 but wherein the data used for each region is defined as follows:

Region I: 0<t<t₁

Region II: 0<t<t₂

Region III: 0<t<t₃

Region IV: 0<t<t₄

Region V: 0<t<t₅

Region VI: 0<t<t₆,

i.e., for each new region analyzed, all prior height vs. time data is used. It should be noted that in the first region, Region I, the blood viscosity calculated using the data 0<t<t₁ is a freshly shed, high shear blood viscosity. It is also desirable to obtain viscosity data in a shear rate range >100 s⁻¹. It should also be noted that the blood viscosity calculated using Region VI data contains the most significant effect of coagulation/clotting because while the columns of blood in riser tubes R1 and R2 fall and rise, respectively, the blood is exposed to air. Thus, the h₁(t) and h₂(t) data/curves contain information about the blood's coagulability characteristics. This segmentation of these data/curves and the subsequent analysis helps further define these coagulability characteristics of the blood.

Based on the above, FIGS. 18A and 18B provide the various blood viscosity profiles (in a log viscosity vs. log shear rate plot) over the six regions, for two hypothetical patients: patient A (FIG. 18A) and patient B (FIG. 18B). By examining the spread in the blood viscosity profiles, one can make a judgment in terms of diagnostics and treatment. For example, patient A shows almost Newtonian type high shear viscosity (Region I viscosity profile is substantially horizontal, i.e., the viscosity in that region does not vary over shear rate). Thus, since it is now possible to identify the blood viscosity profile in the high shear rate range, it is possible to develop and test drugs that alter the living being's blood viscosity to achieve such Newtonian-like performance at high shear rates.

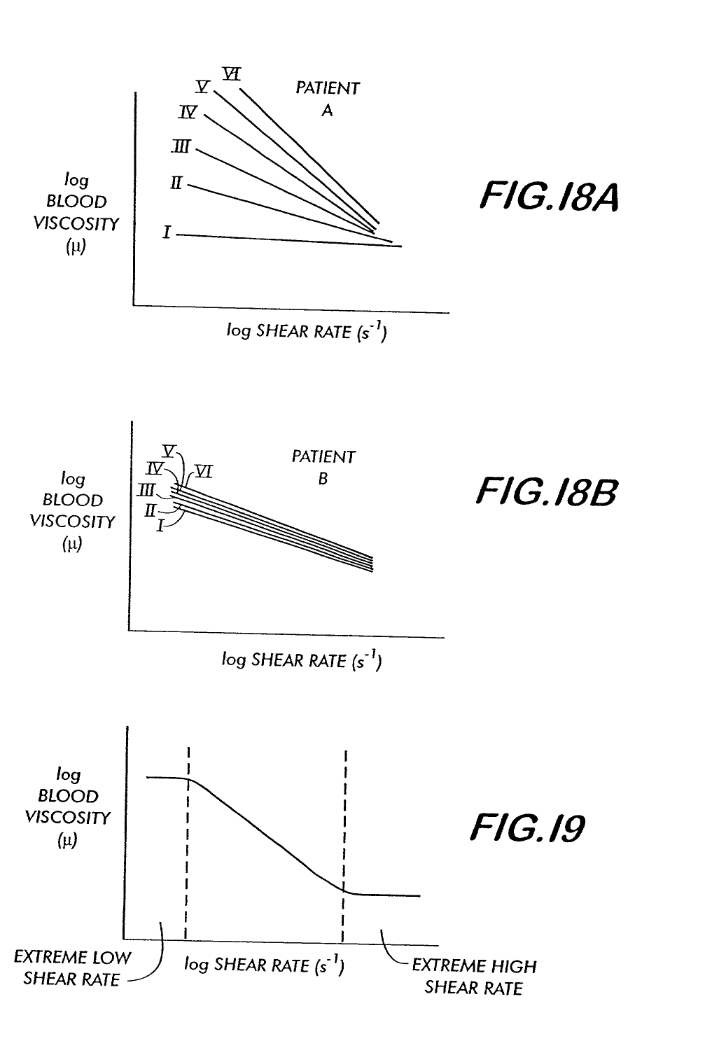

FIG. 19 confirms that the plurality of blood profiles depicted in FIGS. 18A and 18B form the central portion of the log viscosity vs. log shear rate plots, i.e., the extreme ends, both extreme high shear rates and extreme low shear rates, are not plotted or used.

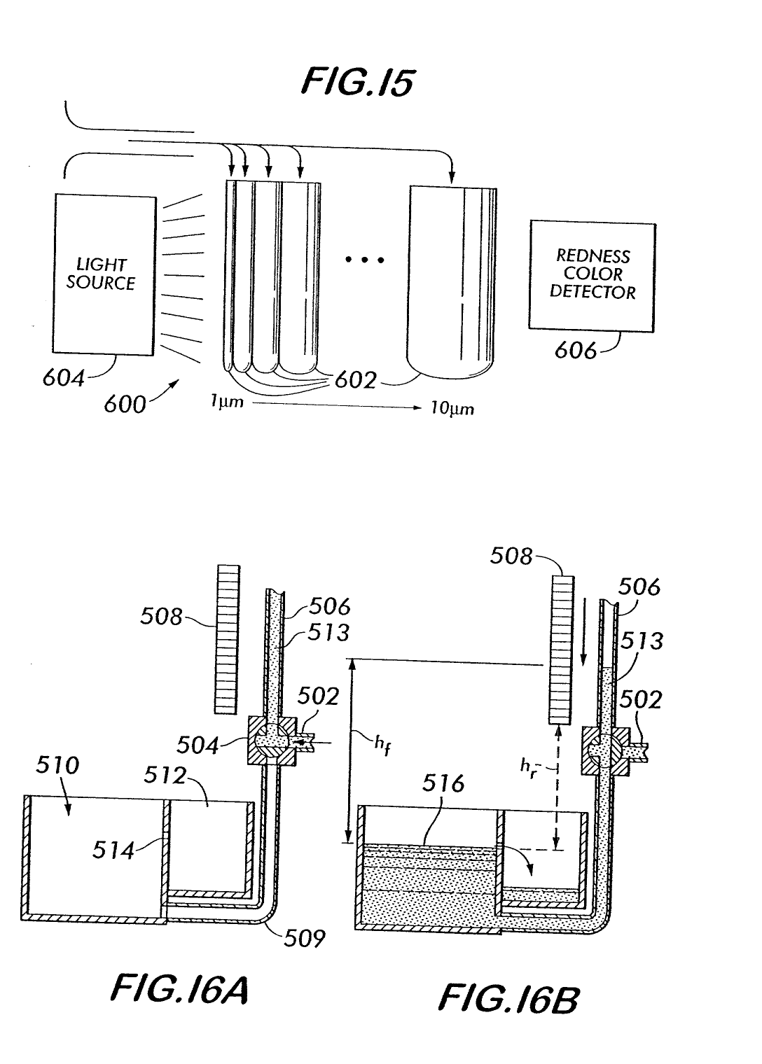

The surface tension analyzer 500 (FIGS. 16A-16B) provides a measurement of the surface tension of a liquid; in the preferred embodiment blood is the liquid whose surface tension is being determined. Typically, surface tension is measured using a bubble-blowing method; however, this method is labor-intensive and a time-consuming procedure. When liquid becomes hazardous to handle, e.g., human blood, it is desirable to have a fully automated procedure.

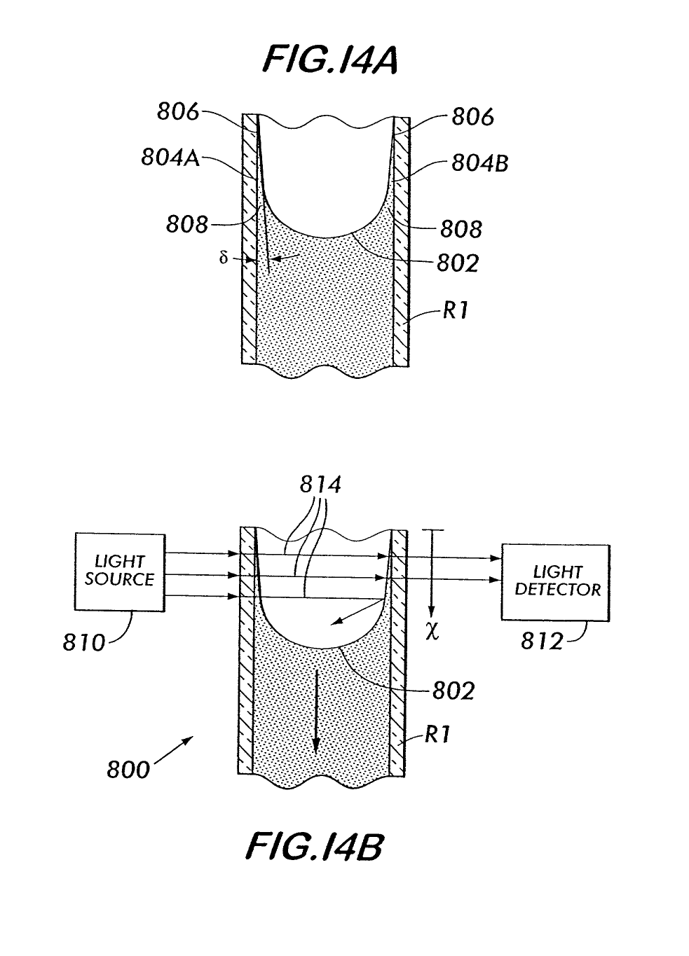

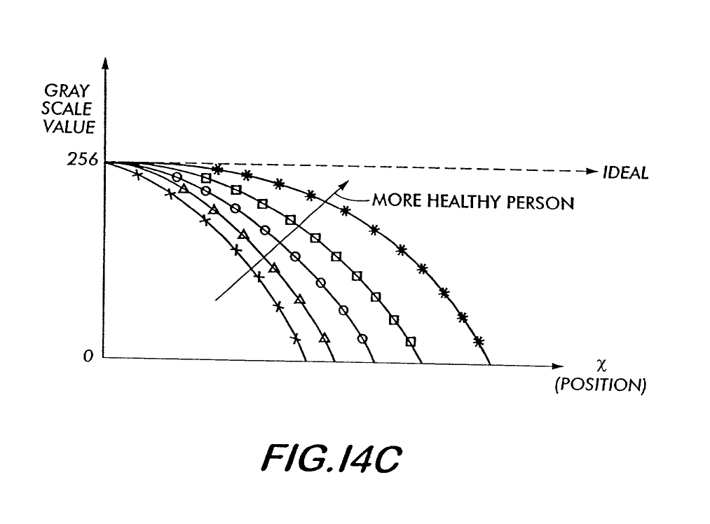

In general, where the surface tension of a liquid is being determined using a cylindrical capillary tube, the surface tension is defined as that upward vertical force which balances the weight of the liquid element. In most liquid/solid interfaces, a contact angle, δ, is formed between the liquid and the capillary tube, such as that depicted in FIG. 14A. However, where the liquid whose surface tension is being determined is water or blood, the contact angle δ=0° and therefore the vertical component of surface tension, namely, πd σ cos δ which counteracts the weight of the liquid, is simply πd σ. Thus, the surface tension, σ, is calculated based on the following:

$$π d σ = ρ ( \frac{π {d}^{2}}{4} h ) g , \\ σ = \frac{ρ dhg}{4}$$

where

σ=surface tension (N/m)

d=capillary tube inside diameter (m); and

h=the height of the liquid element (m)

The surface tension analyzer 500 provides a unique manner for accurately determining the height of the liquid element in the surface tension analysis using capillary rise, as is discussed below.

As shown in FIG. 16A, the surface tension analyzer 500 comprises a conduit 502 from the valve 700, a stopcock 504, a capillary tube 506, a CCD sensor array 508, an elbow 509, a mini-reservoir 510 and an adjacent overflow chamber 512. An aperture 514 is provided in one of the walls of the mini-reservoir 510 adjacent the overflow chamber 512. As will be discussed below, as the column of blood 513 flows down the capillary tube 506, through the stopcock 504, through the elbow 509 and into the mini-reservoir 510, the aperture 514 controls the blood level 516 in the mini-reservoir 510, i.e., as the collected blood level 516 rises to or above the aperture 514, blood flows into the overflow chamber 512. As a result, the exact level of blood in the mini-reservoir 510 is maintained. The CCD sensor array 508 is positioned at a predetermined height, h_{r,} above the aperture 514. As is discussed below, when the CCD sensor array 508 detects the final position of the column in the capillary tube 506, the predetermined height, hᵣ, can be programmed into the CCD sensor array 508 as an offset such the height necessary for the surface tension calculation, namely, h_{f}, is sent to the processor 58. In the alternative, the predetermined height, hᵣ, can already be stored in the processor 58 and only the final position of the column in the capillary tube 508 is detected and transmitted by the CCD sensor array 508 to the processor 58; the processor 58 then adds the value hᵣ to the final position of the column height to arrive at h_{f}. In either case, the processor 58 calculates the surface tension according to the above equation.

The surface tension analyzer 500 operates as follows: Before the test is run, the inside of the capillary tube 506 is wetted by the blood of the living being as it flows from the valve 700. In particular, with the stopcock valve 504 in its initial position as shown in FIG. 16A, the blood flows upward into the capillary tube 506, whose top (not shown) is vented to atmosphere. When the CCD sensor array 508 detects a predetermined level (not shown) of the column of blood 513 in the capillary tube 506, the stopcock valve 504 is rotated to isolate the capillary tube 506 from the conduit 502 and to couple the tube 506 to the elbow 509 and mini-reservoir 510. As the stopcock valve 504 connects the capillary tube 506 to the mini-reservoir 510, blood in the capillary tube 506 falls and settles at a level, h_{f}. The CCD sensor 508 monitors the final position of the column 513. It should be noted that h_{f} is the distance between the blood level in the capillary tube 506 and the level 516 in the mini-reservoir 510 and therefore represents the height of the liquid element required for determining the surface tension, σ, as discussed above. The aperture 514 on the side wall of the mini-reservoir 510 controls the blood level 516 in the reservoir 510. Blood from the reservoir 510 flows into the overflow chamber 512 if the fluid level 516 rises above the aperture 514. Using this level-control aperture, the exact level of blood 516 in the mini-reservoir 510 is known.

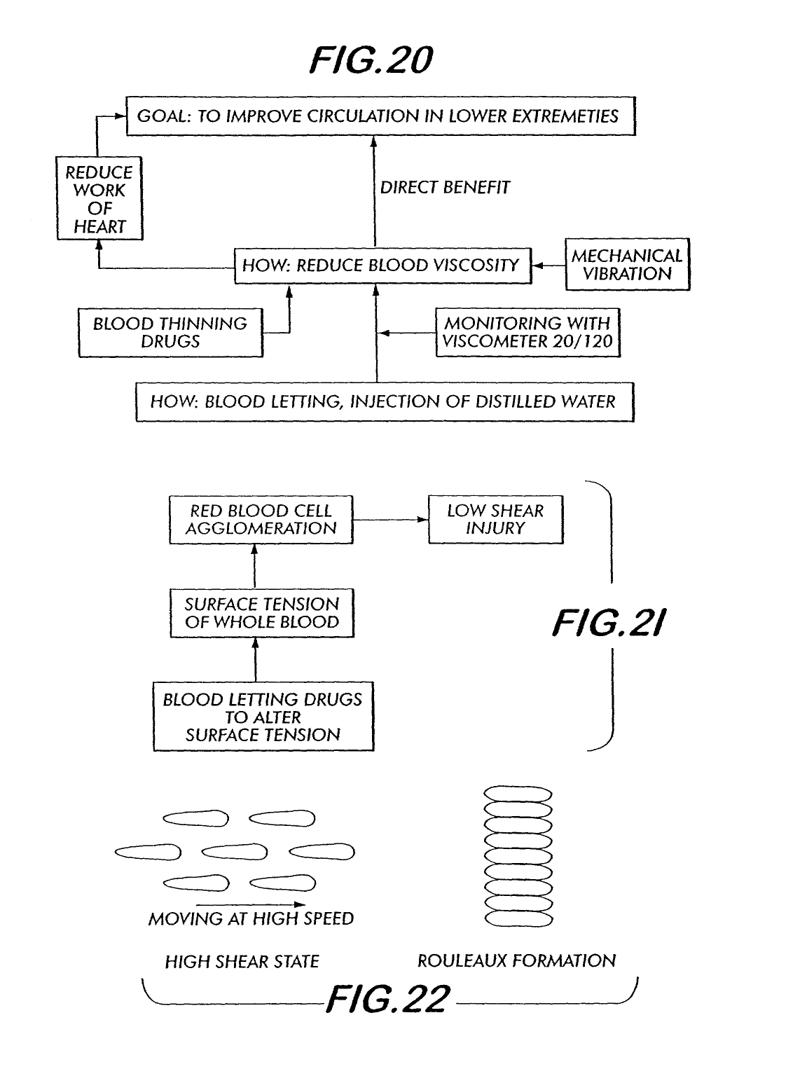

Because surface tension, σ, is related to yield stress, τ₀, (as discussed in application Ser. No. 09/501,856) which is related to RBC (red blood cell) agglomeration (see FIG. 22 where at high shear conditions, the blood cells bonds are evenly spaced allowing these bonds to be easily severed versus low shear conditions where the cells are closely stacked, known as a Rouleaux formation, and where the yield stress/RBC agglomeration causes thrombosis), using the surface tension analyzer 500, it is now possible to determine whether a drug reduces or increases the surface tension of whole blood. In particular, FIG. 21 depicts a method for treating low shear injury through the use of surface tension analyzer 500. The determination of the surface tension of blood can be of great assistance to pharmaceutical companies in their quest to manufacture drugs that reduce the surface tension of whole blood. One of the benefits of reducing the surface tension of blood is to reduce blood viscosity and the work of the heart. For example, saline IV solution and distilled water reduces surface tension and blood viscosity, thus reducing the work of the heart. In addition, blood letting can reduce the surface tension.

Another use of blood viscosity measurement is for treatment of patients with peripheral arterial disease (PAD). Patients with PAD often experience claudication (i.e., lower extremity pain, ache or cramp in the calf, buttock or thigh). PAD occurs when fatty deposits buildup in the arteries, decreasing blood supply to the part of the upper or lower body. This could be due to the insufficient blood flow to the lower extremities. Hence, drugs to reduce peripheral vascular resistance (PVR) are often administered to improve blood flow, thereby reducing pain/ache caused by PAD.

As shown in FIG. 20, a method can be used to improve blood perfusion to the lower extremities by reducing blood viscosity. As mentioned earlier, the reduction of blood viscosity can be accomplished by blood letting or the injection of distilled water (i.e., saline IV solution) or mechanical vibration. By improving circulation and reducing PVR, one can reduce pain while increasing walking distance, as well as quality of life in individuals with intermittent claudication.

Another use of the above methods and apparatus is in the control of hypertension. Typically, there are four basic approaches to control hypertension, each of which is administered independent of the other:

1) β-blocker/calcium-channel blocker which slows down the heart, thereby reducing COH;

2) ACE inhibitor—vasodilator (which opens capillaries in upper/lower extremities);

pure blood pressure lowering drugs;

3) Blood viscosity reduction

blood thinner like Coumadin

Fish oil

Blood letting

Cholesterol-lowering drugs

4) Diuretics—removes water from blood—but actually increases blood viscosity.

In light of the above, a new method of treating hypertension is to apply β-blocker/calcium-channel blocker, ACE inhibitor (including the vasodilator and blood pressure lowering drugs), and blood viscosity reducing drugs in combination to effectively reduce hypertension. The use of diuretics is not to be used with this combination since it has just the opposite effect. Therefore, a pharmaceutical composition combining at least three members selected from the group consisting of β-blockers, calcium-channel blockers, ACE inhibitors (including the vasodilator and blood pressure lowering drugs), and blood viscosity reducing drugs, may reduce the work of the heart, control hypertension and the overall risk of the vascular disease.

The combined use of certain pharmaceutical agents may regulate (ie. alter or maintain) various blood parameters. For instance, the combination of at least two pharmaceutical agents selected from the group consisting of intravenous diluents, red blood cell deformability agents, antiurea agents, oral contraceptives, anti-diabetic agents, antiarrythmics, antihypertensives, antihyperlipidemics, antiplatelet agents, appetite suppressants, anti-obesity agents, blood modifiers, smoking deterrent agents, and nutritional supplements may regulate blood viscosity.

Also, the combination of at least two pharmaceutical agents selected from the group consisting of anti-diabetics, intravenous solutions, cholesterol-lowering agents, triglyceride-lowering agents, lubricants, homocysteine-reducing agents, and vitamin supplements may be used to regulate plasma viscosity.

Additionally, the combination of at least two pharmaceutical agents selected from the group consisting of beta-blockers, calcium channel blockers, ACE inhibitors, ACE-II inhibitors, vasodilators, blood pressure reducing agents, viscosity reducing agents and anti-diabetic agents may regulate the work of the heart.

Also, the combination of at least two pharmaceutical agents selected from the group consisting of beta blockers, calcium channel blockers, ACE inhibitors, ACE-II inhibitors, vasodilators, blood pressure reducing agents, viscosity reducing agents, contractility reducing agents, anti-diabetics, and anti-obesity agents may regulate low shear stress.

Additionally, the combination of at least two pharmaceutical agents selected from the group consisting of intravenous solutions, anti-diabetics, hemodilution agents, anti-platelet agents, lubricity enhancing agents and adhesiveness minimizing agents may regulate high shear stress.

Also, the combination of at least two pharmaceutical agents selected from the group consisting of beta-blockers, calcium channel blockers, and peripheral antiadrenergic/sympatholytics may regulate the contractility of the heart.

Additionally, the combination of at least two pharmaceutical agents selected from the group consisting of anti-thrombogenic agents may regulate the thrombogenicity of the heart.

Also, the combination of at least two pharmaceutical agents selected from the group consisting of warfarin, heparin, and anti-platelet agents (e.g., aspirin) may regulate platelet aggregation.

Additionally, the combination of at least two pharmaceutical agents selected from the group consisting of intravenous fluids, lubricants, anti-adhesives, surfactants, and saponifying agents may regulate lubricity.

Also, the combination of at least two pharmaceutical agents selected from the group consisting of sodium bentonite magma; colloidal clays (such as magnesium bentonite and attapulgite), colloidal silicon dioxide, and microcrystalline cellulose may regulate thixotropy.

Additionally, the combination of at least two pharmaceutical agents selected from the group consisting of gels of colloidal clays, such as sodium bentonite, gels of organic polymers, such as gelatin, agar, pectin, methylcellulose, and high-molecular-weight polyethylene glycol may regulate yield stress.

Also, the combination of at least two pharmaceutical agents selected from the group consisting of beta-blockers and viscosity reducing agents may regulate endothelial shear injury.

Additionally, the combination of at least two pharmaceutical agents selected from the group consisting of anti-thrombogenics and anti-platelets (e.g., aspirin), heparin, and anti-coagulants may regulate coagulability.

Also, the combination of at least two pharmaceutical agents selected from the group consisting of anti-thrombogenics and anti-platelets (e.g., aspirin), heparin, and anti-coagulants may regulate coagulation time.

Additionally, the combination of at least two pharmaceutical agents selected from the group consisting of anti-platelets and anti-coagulants may regulate agglutination.

Also, the combination of at least two pharmaceutical agents selected from the group consisting of anti-thrombogenics and anti-platelets (e.g., aspirin), and anti-coagulants may regulate clot retraction.

Additionally, the combination of at least two pharmaceutical agents selected from the group consisting of anti-thrombogenics and anti-platelets (e.g., aspirin), and anti-coagulants may regulate clot lysis time.

Also, the combination of at least two pharmaceutical agents selected from the group consisting of heparin, warfarin and anti-coagulants may regulate prothrombin rates.

In addition, embodiments of the present method enable adjusting the distribution of a substance through a bloodstream of an organism by altering at least one blood flow parameter of the bloodstream.

The substance being distributed is not particularly limited, but includes, e.g., pharmaceutically active agents.

The method is suitable for use in any organism, including, e.g., single-celled organisms, and multicellular organisms, such as humans and other mammals.

The substance being distributed is suitably administered in any way in which at least some (preferably about 1 wt. % to about 100 wt. %) of the substance reaches the bloodstream of the organism. Thus, the substance can be administered enterally (via the alimentary canal) or parenterally (via any route other than the alimentary canal, such as, e.g., through intravenous injection, subcutaneous injection, intramuscular injection, inhalation percutaneous application, etc.).

Suitable targets for the substance being distributed include, e.g., cells, tissues, organs or systems. Thus, while distribution through the bloodstream is adjusted by this aspect of the invention, the ultimate effects of such adjustments are not limited to the bloodstream specifically or the circulatory system in general. That is, the target for the substance need not be part of the circulatory system, as long as some amount of the substance (in certain embodiments, about 1 wt. % to about 100 wt. % of the substance in the bloodstream) in the bloodstream reaches its intended target.

A distribution parameter for distributing the substance is adjusted, up and/or down, or simply maintained at a desired value, preferably through the use of an agent such as, e.g., levonorgestrel, estrogen, progestin, estradiol, ethinyl estradiol, ethynodiol, medroxyprogesterone, desogestrel, cyproterone, norethindrone, gestodene, norgestrel, mestranol, norgestimate, metformin, acarbose, insulin, chlorpropamide, glipizide, glyburide, tolazamide, glimepiride, troglitazone, pioglitazone, repaglinide, losartan potassium, candesartan cilexetil, irbesartan, mitiglinide, trendolapril/verapamil, nateglinide, nifedipine, nisoldipine, nicardipine, bepridil, isradipine, nimodipine, felodipine, amlodipine, diltiazem, verapamil, isosorbide mononitrate, isosorbide dinitrate, nitroglycerin, hydralazine, minoxidil, hydrochlorothiazide, chlorothiazide, indapamide, metolazone, furosemide, bumetanide, ethacrynic acid, torsemide, spironolactone, triamterene, acetazolamide, mannitol, atenolol, bisoprolol, pindolol, metoprolol, timolol, nadolol, propanolol, carvedilol, captopril, fosinopril, benazepril, lisinopril, enalapril, quinapril, losartan, valsartan, eprosartan, trandolapril, fenoldopam, ramipril, doxazosin, milrinone, benidipine, lemakalim, fantofarone, lemildipine, pirmenol, clentiazem, nebivolol, oxodipine, sematilide, pranidipine, nifekalant, aranidipine, barnidipine, lacidipine, bucindolol, azelnidipine, dofetilide, ibutilide, watanidipine, lercanidipine, landiolol, telmisartan, furnidipine, azimilide, CHF 1521, valsartan/hydrochlorothlazide, enalapril/nitrondipine, sotalol, arbutamine, olmesartan, conivaptan, lovastatin, atorvastatin, cerivastatin, simvastatin, fluvastatin, cholestyramine, colestipol, clofibrate, gemfibrozil, fenofibrate, pamaqueside, pitavastatin, phentermine, phendimetrazine, sibutramine, orlistat, aspirin, warfarin, enoxaparin, heparin, low molecular weight heparin, cilostazol, clopidogrel, ticlopidine, tirofiban, abciximab, dipyridamole, plasma protein fraction, human albumin, low molecular weight dextran, hetastarch, reteplase, alteplase, streptokinase, urokinase, dalteparin, filgrastin, immunoglogulin, ginkolide B, hirudins, foropafant, rocepafant, bivalirudin, dermatan sulfate mediolanum, eptilibatide, thrombomodulin, low molecular weight dermatan sulfate-opocrin, eptacog alfa, argatroban, fondaparinux sodium, tifacogin, lepirudin, desirudin, OP2000, melagatran, roxifiban, parnaparin sodium, human hemoglobin (Hemosol), bovine hemoglobin (Biopure), human hemoglobin (Northfield), antithrombin III, RSR 13, heparin-oral (Emisphere) transgenic antithrombin III, H37695, mesoglycan, CTC111, nicotine, buprorion, fasudil, ziconotide, amino acid preparations, minerals, electrolytes, vitamins, calcitriol, ticarcillin disodium, cefixime, meropenem, cefprozil, levofloxacin, cefpodoxime proxetil, imipenem, cefuroxime axetil, trovafloxacin, mupirocin, stavudine, didanosine, nevirapine, lamivudine, zidovudine, valcyclovir, ganciclovir, nefiracetam, remifentanil, sevoflurane, tiagabine, topiramate, lamotrigine, naratriptan, bromocriptine, tolcapone, oxaprozin, diclofenac, misoprostol, nabumetone, granisetron, dotarizine, RSR13, zonisamide, BMS204352, oxcarbazepine, tropisetron, irinotecan, topetecan, anastrozole, nilutamide, cladribine, gemcitabine, letrozole, vinorelbine, epirubicin, raloxifene, calcitonin, somatotropin, recombinant somatropin, tolterodine, temiverine, meluadrine tartrate, lansoprazole, ropivacaine, bambuterol, israpafant, rupatadine, levosalbutamol, ARC68397AA, salbutamol (powder), salbutamol (inhalation), salbutamol (oral), salbutamol (powder inhilation), formoterol, salmeterol/fluticasone propionate, salmeterol MDI dose counter, salmeterol (inhilation), salmeterol hydrofluoroalkane, budesonide/formoterol, olopatadine, levobetaxolol, levobunolol, latanoprost/timolol, ketotifen, desferoxamine, leukine, sargramostin and GM-CSF.

The distribution parameter can be any such parameter used to evaluate distribution of the substance in an organism. Suitable parameters include, but are not limited to distribution rate and bioavailability.

In embodiments where the distribution rate is decreased, it is particularly preferred to hinder the distribution of a substance, such as a psychoactive ingredient in an addictive product. Another example of such an embodiment is comprises hindering the distribution of toxins and other substances in cigarettes and other tobacco products.

FIG. 15 depicts the red blood cell deformability analyzer 600. In particular, the analyzer 600 comprises a plurality of tubes 602 having various inner diameters in the range from 1 μm to 10 μm and with each tube 602 being in contact with its neighboring tube 602. When circulating blood enters the analyzer 600 from the valve 700, depending on the size of a particular red blood cell (RBC), each RBC will either (1) enter that tube 602 which is large enough for the RBC to pass through, or (2) enter that tube 602 for which the RBC is able to deform without rupturing. As the RBCs collect in various tubes 602, a light source 604 illuminates the plurality of tubes 602. The light passing through the tubes 602 having varying degrees of “redness” is detected by a red color detector 606 (e.g., light source 604/color detector 606 can be implemented by the CS64A color sensor manufactured by Delta Computer Systems, Inc. of Vancouver, Wash. which comprises both a light generation system and a light receiving system for detecting color; a digital/analog converter is used to make the output of the CS64A compatible for computer processing). The higher degree of redness, the higher the RBC content in the tube 602. The red color detector 606 collects the redness information along with the corresponding tube 602 and then passes this information to the processor 58.

FIGS. 14A-14B depict another blood characteristic detector: a blood lubricity detector 800.

It should be understood that although the term “lubricity” is known by those skilled in the mechanical arts as describing the slipperyness between two solids, the term “lubricity” as used in this patent application refers to the “slipperyness” of the blood flow with respect to the vessel wall, i.e., the slipperyness between a liquid (blood) and a solid (the vessel wall).

Furthermore, it should also be understood that another parameter of the blood is the blood's “adhesiveness” which refers to the property of the blood which causes it to cling to the vessel walls. The lubricity and the adhesiveness of the blood are inversely related, namely, as adhesiveness increases the lubricity decreases, and vice versa.

In particular, as shown in FIG. 14A, a meniscus 802 forms at the top of the column of blood as it falls down the riser tube R1. As the meniscus 802 falls, a thin residue of blood of varying thickness is left on the inside surface of the riser tube R1; this is indicated by the reference numbers 804A and 804B. As can be seen, the residue has a minimum thickness at the higher elevations 806 of the riser tube R1 and maximum thickness closest to the meniscus 802 itself, as indicated by the reference number 808. The amount of this residue is indicative of the lubricity of the blood and is exemplary of the lubricity of the blood as it travels through the vascular system of a living being.

To measure this varying amount of film, the lubricity detector 800 is used. The detector 800 comprises a light source 810 located on one side of the risertube R1 near its top portion. The detector 800 also comprises a light detector 812 located on the opposite side of the top portion of the riser tube R1, directly opposite the light source 810. Depending on the thickness of the thin film layer, light rays 814 emanating from the light source will pass through the riser tube R1 wall, a portion of the film of blood on one side of the tube R1, the other thin layer of blood on the opposite side of the tube R1 and through the other side of the riser tube R1 to be detected by the light detector 812. As the residue gets thicker, eventually the light rays 814 directed at that portion of the residue cannot pass through and, as result, are not detected by the detector 812.

An example of the light detector 812 is a CCD having a vertical array of pixels (or an active-pixel sensor (APS) comprising rows/columns of pixels). Light rays 814 that pass through the blood residue in the riser tube R1 impact the pixels at a certain illumination level for different height levels (x) depending on the thickness of the blood residue. If a Gray scale is used to designate varying degrees of illumination (e.g., 256=full light intensity detected; 0=no light detected at all) such pixel data is transmitted to the processor 58 which plots all of these Gray scale values as a function of the vertical position, x. FIG. 14C depicts such plots for different living beings. The processor 58 determines the slope of each curve which is an indicator of the lubricity of a particular living being's blood. An alternative indicator of lubricity may comprise the sum of Gray scale values over a specified vertical length; the higher the sum value, the greater the lubricity since there is little or no blood residue blocking the light rays 814. In a normal healthy living being the lubricity should be high so that a minimum amount of residue, having a minimum thickness, would be left on the inside of the riser tube R1.

It should be understood that blood pressure monitors such as the implantable blood pressure monitors disclosed in U.S. Pat. No. 6,015,386 (Kensey et al.), whose entire disclosure is incorporated by reference herein, or any other type of blood pressure monitor, can be used in combination with the viscometers 20/120 as shown in FIGS. 1 and 2 to accomplish the methods described herein. For example, the implantable blood pressure monitors of U.S. Pat. No. 6,015,386 (Kensey et al.) can be implanted in the living UT and can be used in determining the COH and/or generating the BPN, both of which are discussed above.

The above-described apparatuses and diagnostic methods enable the practice of a variety of prophylactic and/or therapeutic methods using a variety of prophylactic and/or therapeutic compositions to control at least one property of blood measured by the apparatus and methods of the invention.

Table 1, below, provides examples of preferred pharmaceuticals for adjusting blood flow parameters.

| ACTION | THERAPEUTIC/PROPHYLACTIC* |
|---|---|
| Decreasing | cholesterol lowering agents, fish oils, |
| Blood Viscosity | blood thinning agents, intravenous |
| | diluents (e.g., saline, deionized water), |
| | red blood cell deformability agents, |
| | anti-diabetics, anti-urea agents |
| Decreasing | anti-diabetics, intravenous solutions, |
| Plasma Viscosity | cholesterol-lowering agents, |
| | triglyceride-lowering agents, lubricants, |
| | homocysteine-reducing agents, vitamin |
| | supplements |
| Decreasing Work | beta-blockers, calcium channel blockers, |
| of Heart | ACE inhibitors, ACE-II inhibitors, |
| | vasodilators, blood pressure reducing |
| | agents, viscosity reducing agents and |
| | anti-diabetics |
| Decreasing Low | beta blockers, calcium channel blockers, |
| Shear Stress | ACE inhibitors, ACE-II inhibitors, |
| | vasodilators, blood pressure reducing |
| | agents (see above), viscosity reducing |
| | agents, contractility reducing agents, |
| | anti-diabetics, anti-obesity agents |
| Decreasing High | intravenous solutions, anti-diabetics, |
| Shear Stress | hemodilution agents, anti-platelet agents, |
| | lubricity enhancing agents and |
| | adhesiveness minimizing agents. |
| Reducing | beta-blockers; calcium channel blockers; |
| Contractility | peripheral antiadrenergic/sympatholytics |
| of Heart | |
| Reducing | anti-thrombogenics |
| Thrombogenicity | |
| Reducing Platelet | Warfarin, Heparin, Aspirin |
| Aggregation | |
| Increasing | intravenous fluids, lubricants, |
| Lubricity | anti-adhesives, su rfactants, saponifying |
| | agents |
| Altering Thixotropy | Sodium bentonite magma; colloidal clays |
| | (magnesium bentonite, attapulgite); |
| | colloidal silicon dioxide; microcrystalline |
| | cellulose |
| Decreasing Yield | gels of colloidal clays (sodium bentonite); |
| Stress | gels of organic polymers (gelatin, agar, |
| | pectin, methylcellulose, and high- |
| | molecular-weight polyethylene glycol) |
| Reducing Endothelial | beta-blockers, viscosity reducing agents |
| Shear Injury | (see above) |
| Altering | anti-thrombogenics and anti-platelets |
| Coagulability | (e.g., aspirin); Heparin; anti-coagulants |
| Altering Coagulation | anti-thrombogenics and anti-platelets |
| Time | (e.g., aspirin); Heparin; anti-coagulants |
| Altering | anti-platelets; anti-coagulants |
| Agglutination | |
| Altering Clot | anti-thrombogenics and anti-platelets |
| Retraction | (e.g., aspirin); anti-coagulants |
| Altering Prothrombin | Heparin; Warfarin; anti-coagulants |
| Rate | |
| Controlling | beta-blockers, calcium channel blockers, |
| Hypertension | ACE inhibitors, blood pressure reducing |
| | agent and a blood viscosity reducing |
| | agent |

* The following class definitions are in accordance with conventional definitions employed by, e.g., the 12th Edition of the Merck Index (1996) and/or the 54th Edition of the Physician's Desk Reference (1999) and/or the 54th Edition of the Drug Facts and Comparisons (2000) and/or the 19th Edition of Remington's Pharmaceutical Sciences (1995), and are intended to encompass presently existing and subsequently discovered pharmaceuticals listed in these classes.

Without further elaboration, the foregoing will so fully illustrate our invention and others may, by applying current or future knowledge, readily adapt the same for use under various conditions of service.

1. A method to distribute and administer a substance through a bloodstream of an organism, said method comprising:
monitoring at least one blood flow parameter of said bloodstream, said at least one blood flow parameter being selected from the group consisting of circulating blood viscosity, absolute viscosity, effective viscosity, low shear viscosity, high shear viscosity, shear rate of circulating blood, work of heart, contractility of heart, thrombogenicity, platelet aggregation, lubricity, red blood cell deformability, thixotropy, yield stress, coagulability, coagulation time, agglutination, clot retraction, clot lysis time, sedimentation rate and prothrombin rate;
administering said substance to said organism such that an amount of said substance enters said bloodstream; and
distributing at least a portion of said amount of said substance to at least one target within said organism,
wherein a distribution parameter of said distributing is adjusted by altering said at least one blood flow parameter.

2. The method of claim 1, wherein said substance is a pharmaceutically active agent.

3. The method of claim 1, wherein said organism is a human.

4. The method of claim 1, wherein said administering is enteral.

5. The method of claim 1, wherein said administering is parenteral.

6. The method of claim 5, wherein said administering is through intravenous injection, subcutaneous injection, intramuscular injection, inhalation or percutaneous application.

7. The method of claim 1, wherein said amount of said substance is about 1 wt. % to about 100 wt. % of a total amount of said substance administered to said organism.

8. The method of claim 1, wherein said portion is about 1 wt. % to about 100 wt. % of said amount.

9. The method of claim 1, wherein said at least one target is a cell, tissue organ or system.

10. The method of claim 1, wherein said distribution parameter is a rate of said distributing.

11. The method of claim 9, wherein said rate of said distributing is increased.

12. The method of claim 9, wherein said rate of said distributing is decreased.

13. The method of claim 9, wherein said rate of said distributing is decreased and said substance is a psychoactive ingredient of an addictive product.

14. The method of claim 9, wherein said rate of said distributing is decreased and said substance is an ingredient of a tobacco product.

15. The method of claim 1, wherein said altering comprises delivering to said bloodstream an agent effective to alter said at least one blood flow parameter.

16. The method of claim 1, wherein said agent is at least one member selected from the group consisting of levonorgestrel, estrogen, progestin, estradiol, ethinyl estradiol, ethynodiol, medroxyprogesterone, desogestrel, cyproterone, norethindrone, gestodene, norgestrel, mestranol, norgestimate, metformin, acarbose, insulin, chlorpropamide, glipizide, glyburide, tolazamide, glimepiride, troglitazone, pioglitazone, repaglinide, losartan potassium, candesartan cilexetil, irbesartan, mitiglinide, trendolapril/verapamil, nateglinide, nifedipine, nisoldipine, nicardipine, bepridil, isradipine, nimodipine, felodipine, amlodipine, diltiazem, verapamil, isosorbide mononitrate, isosorbide dinitrate, nitroglycerin, hydralazine, minoxidil, hydrochlorothiazide, chlorothiazide, indapamide, metolazone, furosemide, bumetanide, ethacrynic acid, torsemide, spironolactone, triamterene, acetazolamide, mannitol, atenolol, bisoprolol, pindolol, metoprolol, timolol, nadolol, propanolol, carvedilol, captopril, fosinopril, benazepril, lisinopril, enalapril, quinapril, losartan, valsartan, eprosartan, trandolapril, fenoldopam, ramipril, doxazosin, milrinone, benidipine, lemakalim, fantofarone, lemildipine, pirmenol, clentiazem, nebivolol, oxodipine, sematilide, pranidipine, nifekalant, aranidipine, barnidipine, lacidipine, bucindolol, azelnidipine, dofetilide, ibutilide, watanidipine, lercanidipine, landiolol, telmisartan, furnidipine, azimilide, CHF 1521, valsartan/hydrochlorothlazide, enalapril/nitrondipine, sotalol, arbutamine, olmesartan, conivaptan, lovastatin, atorvastatin, cerivastatin, simvastatin, fluvastatin, cholestyramine, colestipol, clofibrate, gemfibrozil, fenofibrate, pamaqueside, pitavastatin, phentermine, phendimetrazine, sibutramine, orlistat, aspirin, warfarin, enoxaparin, heparin, low molecular weight heparin, cilostazol, clopidogrel, ticlopidine, tirofiban, abciximab, dipyridamole, plasma protein fraction, human albumin, low molecular weight dextran, hetastarch, reteplase, alteplase, streptokinase, urokinase, dalteparin, filgrastin, immunoglogulin, ginkolide B, hirudins, foropafant, rocepafant, bivalirudin, dermatan sulfate mediolanum, eptilibatide, thrombomodulin, low molecular weight dermatan sulfate-opocrin, eptacog alfa, argatroban, fondaparinux sodium, tifacogin, lepirudin, desirudin, OP2000, melagatran, roxifiban, parnaparin sodium, human hemoglobin (Hemosol), bovine hemoglobin (Biopure), human hemoglobin (Northfield), antithrombin III, RSR 13, heparin-oral (Emisphere) transgenic antithrombin III, H37695, mesoglycan, CTC111, nicotine, buprorion, fasudil, ziconotide, amino acid preparations, minerals, electrolytes, vitamins, calcitriol, ticarcillin disodium, cefixime, meropenem, cefprozil, levofloxacin, cefpodoxime proxetil, imipenem, cefuroxime axetil, trovafloxacin, mupirocin, stavudine, didanosine, nevirapine, lamivudine, zidovudine, valcyclovir, ganciclovir, nefiracetam, remifentanil, sevoflurane, tiagabine, topiramate, lamotrigine, naratriptan, bromocriptine, tolcapone, oxaprozin, diclofenac, misoprostol, nabumetone, granisetron, dotarizine, RSR13, zonisamide, BMS204352, oxcarbazepine, tropisetron, irinotecan, topetecan, anastrozole, nilutamide, cladribine, gemcitabine, letrozole, vinorelbine, epirubicin, raloxifene, calcitonin, somatotropin, recombinant somatropin, tolterodine, temiverine, meluadrine tartrate, lansoprazole, ropivacaine, bambuterol, israpafant, rupatadine, levosalbutamol, ARC68397AA, salbutamol (powder), salbutamol (inhalation), salbutamol (oral), salbutamol (powder inhilation), formoterol, salmeterol/fluticasone propionate, salmeterol MDI dose counter, salmeterol (inhilation), salmeterol hydrofluoroalkane, budesonide/formoterol, olopatadine, levobetaxolol, levobunolol, latanoprost/timolol, ketotifen, desferoxamine, leukine, sargramostin and GM-CSF.

17. The method of claim 15, wherein said blood flow parameter is blood viscosity and said agent is at least one member selected from the group consisting of intravenous diluents, red blood cell deformability agents, antiurea agents, oral contraceptives, anti-diabetic agents, antiarrythmics, antihypertensives, antihyperlipidemics, antiplatelet agents, appetite suppressants, antiobesity agents, blood modifiers, smoking deterrent agents, and nutritional supplements.

18. The method of claim 15, wherein said blood flow parameter is plasma viscosity and said agent is at least one member selected from the group consisting of anti-diabetics, intravenous solutions, cholesterol-lowering agents, triglyceride-lowering agents, lubricants, homocysteine-reducing agents, and vitamin supplements.

19. The method of claim 15, wherein said blood flow parameter is work of the heart and said agent is at least one member selected from the group consisting of beta-blockers, calcium channel blockers, ACE inhibitors, ACE-II inhibitors, vasodilators, blood pressure reducing agents, viscosity reducing agents and anti-diabetic agents.

20. The method of claim 15, wherein said blood flow parameter is low shear stress and said agent is at least one member selected from the group consisting of beta blockers, calcium channel blockers, ACE inhibitors, ACE-II inhibitors, vasodilators, blood pressure reducing agents, viscosity reducing agents, contractility reducing agents, anti-diabetics, and anti-obesity agents.

21. The method of claim 15, wherein said blood flow parameter is high shear stress and said agent is at least one member selected from the group consisting of intravenous solutions, anti-diabetics, hemodilution agents, anti-platelet agents, lubricity enhancing agents and adhesiveness minimizing agents.

22. The method of claim 15, wherein said blood flow parameter is contractility of the heart and said agent is at least one member selected from the group consisting of beta-blockers, calcium channel blockers, and peripheral antiadrenergic/sympatholytics.

23. The method of claim 15, wherein said blood flow parameter is thrombogenicity of the heart and said agent comprises at least one anti-thrombogenic agent.

24. The method of claim 15, wherein said blood flow parameter is platelet aggregation and said agent is at least one member selected from the group consisting of warfarin, heparin, and anti-platelet agents.

25. The method of claim 15, wherein said blood flow parameter is lubricity and said agent is at least one member selected from the group consisting of intravenous fluids, lubricants, anti-adhesives, surfactants, and saponifying agents.

26. The method of claim 15, wherein said blood flow parameter is thixotropy and said agent is at least one member selected from the group consisting of sodium bentonite magma, colloidal clays, colloidal silicon dioxide, and microcrystalline cellulose.

27. The method of claim 15, wherein said blood flow parameter is yield stress and said agent is at least one member selected from the group consisting of gels of colloidal clays, such as sodium bentonite, gels of organic polymers, such as gelatin, agar, pectin, methylcellulose, and high-molecular-weight polyethylene glycol.

28. The method of claim 15, wherein said blood flow parameter is endothelial shear injury and said agent is at least one member selected from the group consisting of beta-blockers and viscosity reducing agents.

29. The method of claim 15, wherein said blood flow parameter is coagulability and said agent is at least one member selected from the group consisting of anti-thrombogenics, anti-platelets, heparin, and anti-coagulants.

30. The method of claim 15, wherein said blood flow parameter is coagulation time and said agent is at least one member selected from the group consisting of anti-thrombogenics and anti-platelets, heparin, and anti-coagulants.

31. The method of claim 15, wherein said blood flow parameter is agglutination and said agent is at least one member selected from the group consisting of anti-platelets and anti-coagulants.

32. The method of claim 15, wherein said blood flow parameter is clot retraction and said agent is at least one member selected from the group consisting of anti-thrombogenics, anti-platelets and anti-coagulants.

33. The method of claim 15, wherein said blood flow parameter is clot lysis time and said agent is at least one member selected from the group consisting of anti-thrombogenics, anti-platelets and anti-coagulants.

34. The method of claim 15, wherein said blood flow parameter is prothrombin rate and said agent is at least one member selected from the group consisting of heparin, warfarin and anti-coagulants.

35. In a method for distributing a substance through a circulatory system to at least one target in an organism, the improvement wherein at least one blood flow parameter selected from the group consisting of circulating blood viscosity, absolute viscosity, effective viscosity, low shear viscosity, high shear viscosity, shear rate of circulating blood, work of heart, contractility of heart, thrombogenicity, platelet aggregation, lubricity, red blood cell deformability, thixotropy, yield stress, coagulability, coagulation time, agglutination, clot retraction, clot lysis time, sedimentation rate and prothrombin rate is monitored and altered to control said distributing.

36. A composition for administration to an organism having a circulatory system, said composition comprising:
a pharmaceutically active agent; and
a distribution agent effective to increase or decrease distribution of said pharmaceutically active agent through said circulatory system by increasing or decreasing at least one blood flow parameter selected from the group consisting of circulating blood viscosity, absolute viscosity, effective viscosity, low shear viscosity, high shear viscosity, shear rate of circulating blood, work of heart, contractility of heart, thrombogenicity, platelet aggregation, lubricity, red blood cell deformability, thixotropy, yield stress, coagulability, coagulation time, agglutination, clot retraction, clot lysis time, sedimentation rate and prothrombin rate,
wherein said distribution agent is not a diluent.
